(19) United States
(12) Patent Application Publication
(10) Pub. No.: US 2024/0299076 A1
(43) Pub. Date: Sep. 12, 2024

(54) CYCLED PULSING TO MITIGATE THERMAL DAMAGE FOR MULTI-ELECTRODE IRREVERSIBLE ELECTROPORATION THERAPY

(71) Applicants: Virginia Tech Intellectual Properties, Inc., Blacksburg, VA (US); AngioDynamics, Inc., Latham, NY (US)

(72) Inventors: Timothy J. O'Brien, Blacksburg, VA (US); Robert E. Neal, II, Richmond, VA (US); Rafael V. Davalos, Aptos, CA (US)

(21) Appl. No.: 18/608,958

(22) Filed: Mar. 19, 2024

Related U.S. Application Data
(63) Continuation of application No. 16/915,760, filed on Jun. 29, 2020, now Pat. No. 11,950,835.
(60) Provisional application No. 62/892,636, filed on Aug. 28, 2019.
(60) Provisional application No. 62/868,235, filed on Jun. 28, 2019.

(51) Int. Cl. A61B 18/12; A61B 17/00; A61B 18/00; A61B 18/14; A61B 90/00; A61N 1/32

(52) U.S. Cl. CPC A61B 18/1233; A61B 2017/00159; A61B 2017/00172; A61B 2017/0019; A61B 2018/00577; A61B 2018/00613; A61B 2018/00732; A61B 2018/00767; A61B 2018/1253; A61B 2018/126; A61B 2018/1467; A61B 18/1477; A61B 2090/0409; A61N 1/327

(57) ABSTRACT

Methods and systems for distributing electrical energy to tissue which minimize Joule heating, thermal effects, and/or thermal damage, without sacrificing efficacy of treatment, are described. The methods and systems are particularly suitable to electrical energy-based therapies employing multiple electrodes, such as arrays of electrodes.

### CROSS-REFERENCE TO RELATED APPLICATIONS

The present application is a Continuation application of U.S. patent application Ser. No. 16/915,760, filed Jun. 29, 2020, which application relies on the disclosure of and claims priority to and the benefit of the filing date of U.S. Provisional Patent Application No. 62/868,235, filed Jun. 28, 2019 and U.S. Provisional Patent Application No. 62/892,636, filed Aug. 28, 2019, each of which are hereby incorporated by reference in their entireties.

### BACKGROUND OF THE INVENTION

### Field of the Invention

The present invention is directed to the field of treatment of tissue with electrical energy, such as electroporation, or more particularly, such as irreversible electroporation. Embodiments of the invention provide methods and systems for distributing electrical energy to tissue which minimize Joule heating, thermal effects, and/or thermal damage, without sacrificing efficacy of treatment. Embodiments are particularly suitable to electrical energy-based therapies employing multiple electrodes, such as arrays of electrodes.

### Description of Related Art

A variety of electrical energy-based therapies have been developed, including reversible electroporation, electrochemotherapy, electrogenetherapy, supraporation, radiofrequency ablation, irreversible electroporation (IRE) and high frequency irreversible electroporation (HFIRE). It is often desirable to limit tissue hyperthermia during these treatments. While a number of solutions have been proposed, there remains a need for improvements.

### SUMMARY OF THE INVENTION

The present inventors have developed systems and methods which distribute an amount of electrical energy to tissue in a manner which minimizes Joule heating to tissue. While various embodiments will be discussed in the foregoing Detailed Description, it should be understood that these are merely provided to illustrate various implementations, of which variations which are encompassed within the principles of the invention may occur to the skilled artisan reading the benefit of this disclosure.

Embodiments of the invention include Aspect 1, which is a method of treating tissue comprising: disposing a number of electrodes in tissue to outline a target region to be treated; optionally selecting a total number of electrical pulses to be delivered to the target region; and delivering a total number of electrical pulses to the target region by: activating a number of pairs of the electrodes in a number of cycles; wherein the activating is performed a number of times that equals the total number of electrical pulses, divided by the number of pairs of electrodes, divided by the number of cycles; whereby electrical energy is strategically distributed to sub-regions within the outline of the target region to treat tissue while mitigating one or more of thermal effects or damage, potential for Joule heating, or delivery of electric current to tissue of the target region.

Aspect 2, is the method of Aspect 1, wherein each pair of electrodes delivers a pulse train with no delay between pulses in the pulse train.

Aspect 3 is the method of Aspect 1 or 2, further comprising adding one or more delay between electrical pulses and/or bursts in a manner to further mitigate one or more of the thermal effects, thermal damage, potential for Joule heating, or delivery of electric current to tissue of the target region.

Aspect 4 is the method of any of Aspects 1-3, wherein the thermal effects or thermal damage are evidenced by an amount of white tissue coagulation.

Aspect 5 is the method of any of Aspects 1-4, wherein a ratio of thermally damaged tissue area to ablation area is less than 5%.

Aspect 6 is the method of any of Aspects 1-5, wherein the number of electrodes is less than the number of pairs of electrodes.

Aspect 7 is the method of any of Aspects 1-6, wherein the activating is performed such that no single electrode is activated more than two consecutive times within a cycle.

Aspect 8 is the method of any of Aspects 1-7, wherein the same and/or different sub-regions of the target region are treated consecutively.

Aspect 9 is the method of any of Aspects 1-8, wherein the activating comprises applying a first pulse train to a first pair of electrodes, applying a second pulse train to a second pair of electrodes, optionally applying additional pulse trains to one or more additional pairs of electrodes, then again applying a pulse train to the first pair of electrodes to start a new cycle, and repeating such activating over the number of cycles until the total number of determined electrical pulses is reached.

Aspect 10 is the method of any of Aspects 1-8, wherein the delivering of the electrical pulses causes electroporation based therapy, electroporation, irreversible electroporation, reversible electroporation, electrochemotherapy, electrogenetherapy, supraporation, and/or high frequency irreversible electroporation, or combinations thereof.

Aspect 11 is the method of any of Aspects 1-10, wherein the delivering causes IRE and/or HFIRE.

Aspect 12 is the method of any of Aspects 1-11, wherein one or more of the following parameters are employed for the delivering of the electrical pulses: a) the number of cycles is from one to ten; b) one or more delays of 0 to 10 seconds each (within a pulse train and/or between activation of pairs and/or between cycles); c) a number of pulses per cycle and/or pulses per pair of 10 to 200; d) a total number of pulses of 100 to 5000; and/or e) a total number of pairs of electrodes of from 3 to 28.

Aspect 13 is the method of any of Aspects 1-12, wherein the delivering is performed: using a voltage ranging from 0 V to 10,000 V; and/or with pulse lengths in the ns to second range; and/or with a frequency in the range of 0 Hz to 100 MHz; and/or with a waveform that is square, triangular, trapezoidal, exponential decay, sawtooth, sinusoidal, and/or alternating polarity; and/or with a total number of pulses ranging from 1-5,000 pulses; and/or with a total number of pulses per pulse train ranging from 1-5,000 pulses.

Aspect 14 is a method of treating a tissue with electrical energy while mitigating thermal damage to the tissue comprising: distributing a total number of electrical pulses to the tissue by way of a plurality of electrode pairs over a plurality of cycles, such that each electrode pair is activated to receive one or more pulse train during each cycle of the plurality of cycles.

Aspect 15 is the method of Aspect 14, wherein each pair of electrodes delivers a pulse train with no delay between pulses in the pulse train.

Aspect 16 is the method of Aspect 14 or 15, further comprising introducing a delay between one or more of the electrical pulses and/or one or more pulse train.

Aspect 17 is an electrical energy based system comprising: an array of three or more electrodes; an electrical pulse generator in operable communication with the electrodes; wherein the electrical pulse generator comprises programming capable of activating a plurality of pairs of electrodes in a manner which distributes an electric field at different spatial coordinates at different times in a manner to mitigate one or more of thermal effects, thermal damage, potential for Joule heating, or delivery of electric current to tissue of the target region.

Aspect 18 is the system of Aspect 17, wherein:
the programming is capable of distributing a total number of pulses over multiple pairs of electrodes,
wherein pairs of electrodes are individually and sequentially activated over multiple cycles such that:
P_{T}=(εp) (P̅_{#}) (C_{#}), wherein:
P_{T}=the total number of pulses delivered to tissue;
εₚ=the total number of electrode pair combinations;
P̅_{#}=the average number of pulses delivered in a pulse train per activated electrode pair per cycle: and
C_{#}=the total number of cycles.

Aspect 19 is the system of Aspect 17 or 18, wherein the programming is capable of introducing one or more delays between one or more of the electrical pulses.

Aspect 20 is the system of any of Aspects 17-19, wherein the one or more delays are introduced within one or more pulse train applied to each pair, and/or between activation of one or more electrode pair, and/or between one or more of the cycles.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a block diagram of an electroporation device according to one aspect of the present invention.

FIG. 2 is a block diagram of a treatment control computer of FIG. 1.

FIG. 3 is a block diagram of a pulse generator shown in FIG. 1.

FIG. 4 is a block diagram of a sensor of FIG. 3.

FIGS. 5A-C are illustrations of pulse delivery methods, where FIG. 5A is an illustration of an exemplary conventional pulse delivery scheme (i.e., non-cycled pulsing) using a conventional EPAP scheme, where 100 pulses were delivered per electrode pair for a total number of 600 pulses to the target tissue. FIG. 5B is an illustration of an embodiment of a cycled pulse paradigm but using the conventional EPAP scheme of FIG. 5A (5 pulse cycle, 0 s delay scheme), where 20 pulses were delivered per electrode pair, yielding 120 total pulses per cycle and, again, a total of 600 pulses to the target region. FIG. 5C is an illustration of an embodiment of the same cycled pulse paradigm shown in FIG. 5B, except that FIG. 5C shows an enhanced electrode pair activation pattern (EPAP), such that no single electrode was activated more than two consecutive times, whereas in FIG. 5B, at least one of the electrodes is activated three times consecutively. Alternatively, instead of pulses, bursts of pulses can be applied, such as instead of 100 pulses, 100 bursts of pulses can be applied, or instead of 20 pulses, 20 bursts of pulses can be applied.

FIG. 6A is a Computer-Aided Design (CAD) rendering of a 4-electrode support device to ensure equidistant spacing throughout each treatment. In embodiments, the electrode spacing of the support device (and thus the spacing of the holes through which the electrodes would pass) can be disposed as shown in FIG. 6A such that the four innermost holes are spaced about 1 cm apart, the four outermost holes are spaced about 2 cm apart, and the four intermediate holes (disposed substantially concentrically between the innermost and outermost holes) are spaced about 1.5 cm apart, with a hole diameter of a size sufficient to accommodate an electrode, such as about 0.24 cm.

FIGS. 6B and 6C are schematic diagrams showing fiber optic thermal sensor placement in the “xy” and “xz” plane, respectively. Each temperature sensor was labeled T1-T4 to understand temperature trends at each electrode.

FIG. 6D is an image showing the implementation of the support devices on the perfused organ model for the 4-monopolar electrode configuration.

FIG. 6E is a schematic diagram of the perfusion, fiber optic thermal measurement, and pulse delivery systems.

FIG. 7A is a schematic diagram showing the placement of four monopolar electrodes. The sliced plane is taken at the midpoint of the IRE ablation depth. One half is used to measure the cross-sectional area. FIG. 7B shows the IRE ablation zone and thermally damaged regions are highlighted.

FIG. 8 is a series of illustrations showing visualization of the electric field distributions for each electrode pair. The combined maximum intensity of each individual field represents the effective electric field distribution from treatment with this four electrode array. This sequence of pulsing represents a conventional pulsing scheme.

FIG. 9 is a graph which illustrates the measured and numerically calculated thermal response for a “10 pulse cycle, 10 s delay” pulsing sequence at electrode 2 (T2). A schematic illustrating fiber optic thermal probe location relative to electrodes is shown in the top right corner of the figure. The average RMSE for each electrode was 6.02° C. for this pulsing paradigm.

FIGS. 10A-F are images showing representative cross-sectional tissue sample images for the conventional paradigm (FIG. 10A), 1 pulse cycle, 0 s delay (FIG. 10B), 5 pulse cycles, 0 s delay (FIG. 10C), 10 pulse cycles, 0 s delay (FIG. 10D), 10 pulse cycles, 5 s delay (FIG. 10E), 10 pulse cycles, 10 s delay (FIG. 10F).

FIGS. 10G-I are graphs showing a comparison between the experimental and numerical results for the percentage ratio of thermal damage as shown by the white tissue coagulation area (i.e., tissue whitening) to IRE treatment zone area (FIG. 10G) (p<0.0001, all pairwise comparisons), the average cross-sectional ablation area for each pulse scheme (FIG. 10H), and the average area of white tissue coagulation (FIG. 10I) (Ω=2.3, p<0.0001, all pairwise comparisons).

FIG. 11A is a graph showing the calculated average energy delivered to the tissue for the amount of time shown in FIG. 11B (the energy deposited was about the same across the groups). FIG. 11B is a graph showing the measured treatment time for each pulse paradigm, where *, #, and % groups are significantly different from one another (p<0.0001, all pairwise comparisons). As shown in FIG. 11B, the protocols with a zero second delay displayed shorter treatment times on average than other pulse schemes, the conventional paradigm and “10 pulse cycle, 5 s delay” group were statistically similar, and the treatment time for the “10 pulse cycle, 10 s delay” group was the longest on average.

FIGS. 12A-12C are graphs showing numerical modeling data of the area of tissue that experiences an electric field greater than 601 V/cm for the conventional and enhanced electrode pair activation patterns (EPAPs) (FIG. 12A) for the various conventional and cycled pulsing protocols indicated along the X axis, the area of tissue that experiences thermal damage (Ω=2.3) for both EPAPs (FIG. 12B) for these protocols, and the percentage ratio of thermal damage area to IRE treatment zone area (FIG. 12C) for these protocols. The initial temperature within the numerical model was set to Tₒ=30° C. to match the experimental settings.

FIGS. 13A-13C are series of illustrations showing the numerically derived thermal distribution at the completion of each initial electrode pair activation, as well as the final electric field and thermal distribution 50 seconds post therapy for a conventional paradigm and conventional EPAP (FIG. 13A), a cycled pulsing paradigm (10 pulse cycles, 5 s delay) with a conventional EPAP (FIG. 13B), a cycled pulsing paradigm (10 pulse cycles, 5 s delay) with an enhanced EPAP (FIG. 13C). The time at each electrode pair activation is displayed in the top left corner. The area in which the onset of thermal damage occurs (Ω=2.3) is encircled in bold. Initial temperature within the numerical model was set to Tₒ=30° C. to match the experimental settings.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

### Definitions

The term “consecutive” activation of a single electrode refers to activation of a specific electrode during pulse delivery through a first electrode pair, followed by activation of the next electrode pair that includes that same specific electrode. Consecutive activation may also be referred to as sequential activation, and consecutive/sequential activation of a single electrode occurs regardless of direction of energy flow between the electrodes within the electrode pair. Said another way, an electrode is consecutively/sequentially activated if it is an electrode of one activated pair and an electrode of a second activated pair that is activated consecutively/sequentially after the first pair, and whether energy is moving from or to the electrode in either pair.

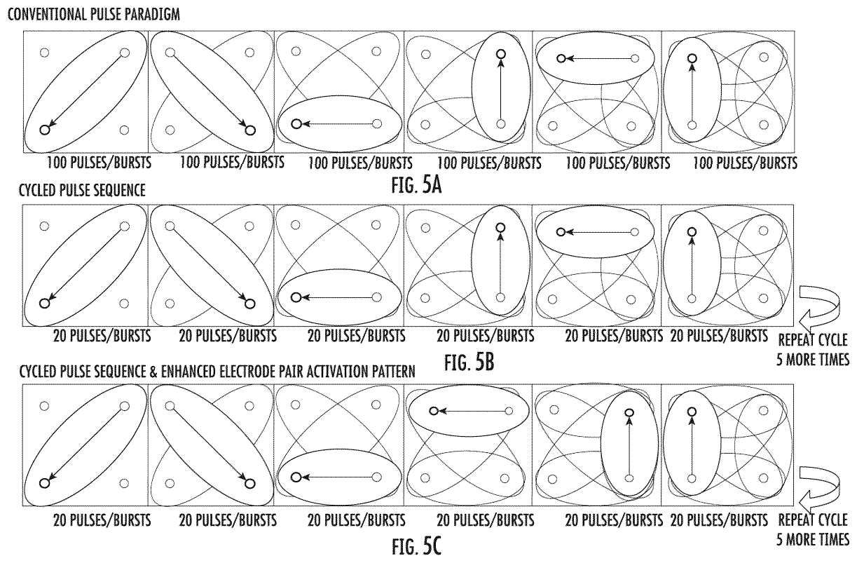

The term “conventional protocol” refers to a pulse scheme in which all pulses are delivered within a single cycle. All pulses for a specific electrode pair are delivered before the next electrode pair is activated. Typically, to determine the total number of pulses to be delivered for a particular treatment, the desired number of pulses per electrode pair can be determined and multiplied by the total number of electrode pairs. Alternatively or in addition, the number of pulses delivered per electrode pair can be calculated by dividing the total number of desired pulses by the number of electrode pairs to be activated. The term may be used interchangeably with “conventional treatment,” “conventional pulsing scheme,” “conventional paradigm,” “conventional pulse scheme,” “conventional pulse paradigm,” “conventional pulse protocol,” “conventional pulsing sequence,” “conventional pulsing paradigm,” “conventional sequence,” or a pulse scheme may just be labeled as “conventional.” For example, a conventional pulse scheme is shown in FIG. 5A.

The term “cycled pulsing scheme” refers to a pulse scheme in which the total number of pulses are delivered over more than one cycle. The total number of pulses per cycle is calculated by dividing the total number of desired pulses by the number of cycles. The term may be used interchangeably with “cycled pulsing protocol,” “cycled pulsing sequence,” “cycled pulsing,” “cycled pulsing paradigm,” “cycled pulsing embodiment,” “cycled pulse sequencing,” “cycled pulse paradigm,” or “cycled pulsing pattern.” For example, a cycled pulsing scheme is shown in FIGS. 5B and 5C.

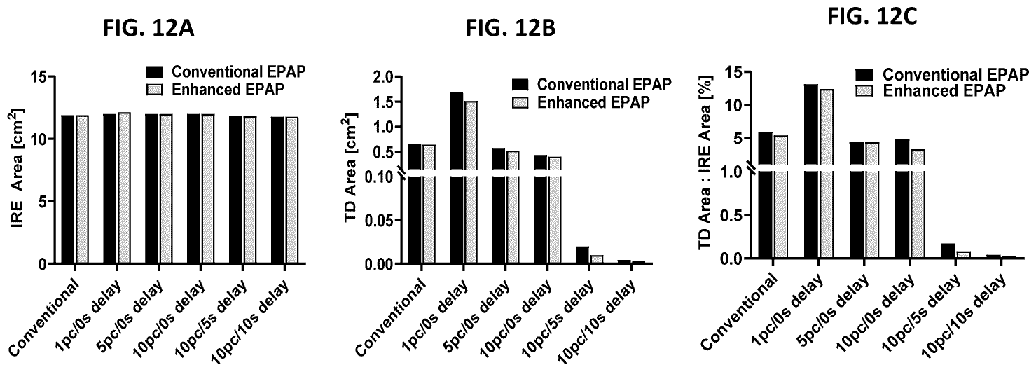

The term “electrode pair activation pattern (EPAP)” refers to the sequence in which electrode pairs are activated within a cycle or across multiple cycles. Each of FIGS. 5A-C shows an EPAP, with FIGS. 5A-B showing conventional EPAP and FIG. 5C showing enhanced EPAP. Further, for example, FIGS. 12A-C show conventional and enhanced EPAP can be used with either or both conventional protocols and cycled pulsing protocols.

The term “conventional EPAP” refers to an electrode pair activation pattern (EPAP) in which pulses are delivered without regard to sequential or consecutive activation of any electrode from one activated electrode pair to the next. With conventional EPAP, an electrode can be activated in a first pair, then the same electrode can be activated again in the next activated pair, and activated again in the next activated pair, such as is shown in FIG. 5A.

The term “enhanced” electrode pair activation pattern (EPAP) refers to an improved electrode pair activation pattern (EPAP) in which sequential or consecutive activation of a single electrode is minimized, as shown in FIG. 5C and FIGS. 12A-C. For example, an electrode is activated in a first pair, then activated in the next activated pair, but is not activated in the next activated pair.

Embodiments of the invention include a method which distributes electrical energy to tissue over geometric space and/or time in a manner which reduces heating, such as Joule heating, of the tissue.

Embodiments also include a method which applies an electric field in a manner that increases the amount of time a quantity of electrical energy is applied to an area or volume of tissue, thereby reducing the concentration of electrical energy applied to the area or volume of tissue per unit time and/or reducing the electric field applied to the area or volume of tissue per unit time.

Advantages resulting from the methods include reduced Joule heating and/or reduced thermal damage to tissue. For example, while the mechanisms for IRE cell death are non-thermal, temperature and electric current variations occur due to high electric field gradients immediately adjacent to the needle electrodes. In one application, the methods are useful for reducing Joule heating and/or thermal damage resulting from irreversible electroporation (IRE).

One embodiment includes a method of treating tissue. The method includes subdividing a total number of multiple electrical pulses to be delivered to tissue, delivering the electrical pulses to the tissue according to the subdividing, and optionally including one or more delay during treatment between one or more of the multiple electrical pulses in a manner to reduce thermal effects or thermal damage, such as white tissue coagulation, and/or to reduce electric current, while maintaining ablation size.

According to embodiments, the total number of electrical pulses is subdivided between a number of electrode pairs and/or subdivided over a number of cycles. Embodiments can also include delays which are added throughout the treatment. The electrical pulses can be of sufficient energy to produce ablation between all electrodes.

Another embodiment includes a method of treating a target region with electrical energy. The method includes disposing one or more electrodes in or near a target region to be treated, and using the electrode(s) to deliver multiple electrical pulses to the target region in a manner such that only some of the electrodes are actively in use at a time. The delivering is performed in a manner to reduce thermal damage and/or to reduce thermal effects and/or to minimize potential for Joule heating and/or to reduce delivery of electric current.

Another embodiment includes a method of treating a target region with electrical energy, where the method includes disposing one or more electrodes in or near a target region to be treated, and delivering multiple electrical pulses to the target region by way of the electrodes in a manner such that the same and/or different areas of the target region are treated consecutively. The delivering is performed in a manner to reduce thermal damage and/or to reduce thermal effects and/or to minimize potential for Joule heating and/or to reduce delivery of electric current.

Another embodiment includes a method of treating a target tissue, the method comprising distributing an amount of electrical energy over a plurality of pairs of electrodes representing different regions of the target tissue over a plurality of cycles in a manner which reduces thermal damage and/or reduces thermal effects and/or minimizes potential for Joule heating and/or reduces an amount of electric current delivered to a region of the target tissue per unit time.

Another embodiment includes a method of treating a tissue with electrical energy while mitigating thermal damage to the tissue. The method includes distributing electrical energy to the tissue over geometric space and time in a manner which reduces an amount of electrical energy a volume of tissue receives per unit time. Distributing electrical energy to the tissue can include activating a plurality of pairs of electrodes implanted within the tissue in a manner which produces a unique electric field distribution in the tissue at different times. Activating a plurality of pairs of electrodes can include applying a first pulse train to a first pair of electrodes, then applying a second pulse train to a second pair of electrodes, and then applying additional pulse trains to one or more additional pairs of electrodes and then again to the first pair of electrodes to start a new cycle, and repeating such activation over cycles until a targeted number of pulses for each pair of electrodes is reached.

Another embodiment includes a method of treating a tissue with electrical energy while mitigating thermal damage to the tissue, where the method includes distributing a total number of pulses to the tissue by way of a plurality of electrode pairs implanted in tissue over a plurality of cycles such that each electrode pair is activated to receive a pulse train over each cycle of the plurality of cycles.

According to embodiments, the total number of pulses is distributed as per Equation 1:

$${P}_{T =} ( {ε}_{p} ) ( {\overline{P}}_{\#} ) ( {C}_{\#} ) \qquad [ 1 ]$$

wherein:
P_{T}=the total number of pulses delivered to tissue
εₚ=the total number of electrode pair combinations
P̅_{#}=the average number of pulses delivered in a pulse train per activated electrode pair per cycle
C_{#}=the total number of cycles.

The total number of pulses or bursts of pulses applied to tissue can be chosen to deliver a targeted quantity of electrical energy to the tissue. In the Example to follow, a protocol of 600 pulses was chosen. However, it can be appreciated that 600 bursts of pulses could be applied instead, and/or that the number of pulses can be chosen to fit a particular application and/or clinical situation for treating tissue with electrical energy. In embodiments, the total number of pulses or bursts of pulses can range in the hundreds or more, such as one hundred to several hundred (over one thousand), depending on the amount of energy the application or clinical situation requires. For example, for ablation applications, naturally larger tumor masses may require more pulses/bursts than smaller tumor masses. The conductivity of different tissue types, or tumor types, may also dictate the amount of energy and/or number of pulses/bursts delivered. The plurality of electrical pulses can have a total number of pulses, a total number of bursts of pulses, and/or a total number of pulses per burst, ranging from 1-5,000 pulses/bursts, such as from at least 1 up to 3,000 pulses/bursts, or at least 2 up to 2,000 pulses/bursts, or at least 5 up to 1,000 pulses/bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby. In any embodiment of the cycled pulsing protocols, it would be understood that the number of pulses is at least 2 or more. The electrical pulses and/or bursts and/or electrical energy and/or electric current can be of an intensity or quantity sufficient for ablating tissue, such as ablation by electroporation, such as IRE or HFIRE.

The total number of electrode pair combinations will depend on the number of electrodes chosen to deliver the pulses to the tissue. In the Example to follow, the four electrode array chosen has a total possible number of electrode pair combinations of six. However, it will be appreciated that other variations are possible. For example, a six electrode array will have a total possible number of electrode pair combinations of fifteen. Table 1 below is illustrative.

TABLE 1

| number of electrodes | possible number of pairs |
|---|---|
| 3 | 3 |
| 4 | 6 |
| 5 | 10 |
| 6 | 15 |
| 7 | 21 |
| 8 | 28 |

The number of pairs of electrodes chosen for delivery of the pulses can be less than the possible number of pairs. For example, the geometry of the electrode array chosen by the skilled artisan may cause some electrode pairs to be positioned to close or too far away from each other for practical delivery of electrical energy to tissue. Also, some pairs may be redundant in that they cover substantially the same area or volume of tissue. The geometry of the array and number of electrodes can be chosen based on, for example, the geometry of the targeted tissue area that one of skill in the art wishes to ablate (e.g. the contours of a tumor mass obtained during imaging, the presence of critical structures near the tumor mass, such as blood vessels or nerves). Again, it can be appreciated that larger tumor masses can require larger arrays than smaller masses. The delivering of the electrical pulses is performed such that pairs of electrodes deliver the electrical pulses and/or bursts of pulses in a manner such that an area of the target region bounded by all of the electrodes is subjected to an electrical field. It is important to note that the electrodes can be monopolar or bipolar electrodes. For example, the electrode pair activation patterns as shown in FIGS. 5A-C could be applied using bipolar electrodes and as such FIGS. 5A-C as shown would represent a side view of two probes inserted into tissue, with each probe comprising two bipolar electrodes. In some cases, electrodes on each probe could be activated to treat the area between the probes and/or electrodes on one probe or the other probe could be activated to treat the area along a probe.

Pulse trains can be delivered or distributed amongst the electrode pairs over several cycles. The particular number of pulses for each pair can be chosen according to the skilled artisan's discretion. While in the Example to follow, 20 pulses or bursts per pair was chosen for the cycled pulsing sequence for each pair, the practitioner need not be limited to using the same number of pulses per pair and/or per cycle. For example, he or she may want to deliver more or less energy to different areas of targeted tissue, and/or at different times, and chose the number of pulses accordingly, again based on such potential factors as features of the tumor, the presence or absence of critical structures, tissue conductivities, and so on. The number of pulses or bursts per pair and number of cycles can be chosen to distribute the total amount of energy chosen (e.g. as total number of pulses) over the geometric area chosen (e.g. size of the electrode array) in a time frame adequate to the application or clinical situation. For example, the number of pulses or bursts for each pair can be in the range of 1 to 100, and the number of cycles can be 1, 2, 3, 4, 5, 6, 7, 8, 9, 10, 11, 12, 13, 14, 15, 16, 17, 18, 19, 20 or more.

The number of pulses and cycles can be chosen to minimize thermal damage delivered during the delivery of electrical energy to tissue. Another advantage of the inventive protocols, and in particular balancing the distribution of pulses/bursts over the treatment area and over the time of the treatment, is a favorable immune response as a result of the ablation (such as IRE) in contrast with treatments using thermal ablation techniques. It has been found that the non-thermal nature of IRE encourages a unique immune response compared to other ablative technologies. For example, it has been shown that IRE could trigger as much as 2-3 times the amount of T cell proliferation in comparison to thermal therapies. SHAO, Qi et al., Engineering T cell response to cancer antigens by choice of focal therapeutic conditions, International Journal of Hyperthermia, 2019, DOI: 10.1080/02656736.2018.1539253. Benefits further include the ability for the practitioner to dispose electrodes/probes closer to critical structures without damaging those structures or causing very little damage to such structures. In treating tumors that are disposed close to critical structures, any distance closer to the structure helps and can lead to better treatment results for the patient, in being able to treat tumors in their entirety instead of only partially, which would be a common result of treating in a manner to avoid damaging nearby critical structures. Another benefit of the inventive pulse/burst protocols is in being able to minimize treatment times. The treatment duration can be less than 1 hour, such as less than any of 45 min., 30 min., 15 min., 10 min., or 5 min., such as less than about 7-17 min., wherein the treatment duration is calculated including or not including any delay.

According to embodiments, the order that pairs of electrodes are activated can be the same over each of the cycles, or can be altered from cycle to cycle or for two or more cycles. Further, the order in which electrode pairs are activated can be adjusted to minimize consecutive electrode activation. For example, the order of electrode pair activation can be adjusted such that no single electrode is activated more than two consecutive times. Such order can be implemented with other features of the pulsing protocol to minimize Joule heating.

According to any embodiment, each pair of electrodes can be activated by a pulse train with no delay between pulses in the pulse train. In other embodiments, one or more delays can be introduced, such as a delay between one or more pulses in the pulse train, and/or a delay between the activation of one or more pair of electrodes, and/or a delay between one or more of the cycles. The delay can be on the order of microseconds or seconds, such as one to one thousand microseconds, such as 1, 2, 3, 4, 5, 6, 7, 8, 9, 10, 15, 20, 25, 30, 35, 40, 45, 50, 75, 100, 150, 200, 250, 300, 350, 400, 450, 500, 600, 700, 800, 900, or 1000 microseconds or one to several seconds such as 1, 1.5, 2, 2.5, 3. 3.5, 4, 4.5, 5, 5.5, 6, 6.5, 7, 7.5, 8, 8.5, 9, 9.5, 10, 11, 12, 13, 14, 15, 16, 17, 18, 19, 20, 22, 24, 26, 28, 30 seconds or more. Cumulatively, the one or more delays may be on the order of seconds or minutes.

According to embodiments, the methods treat a target region or tissue with electrical energy in order to ablate cells in the target region or tissue. The target region or tissue can include cancer or non-cancer cells or both. As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sé zary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor. Benign or non-cancerous tumors or growths for example can include fibroids or fibromas such as uterine fibroids, adenomas such as polyps, lipomas, myomas, nevi or moles, or growth due to benign prostate hyperplasia (BPH), myxoma, papillary fibroclastomas, rhabdomyomas, hemangiomas, teratomas, paragangliomas, cysts such as pericardial cysts or ovarian cysts, warts, endometriosis, and including any abnormal or undesired growth. Other conditions that can be treated using embodiments of the invention include treatment of cardiac arterial fibrillation and fat removal. In one embodiment, the target region or tissue is a solid tumor.

According to embodiments, methods of treating a target region or tissue with electrical energy which distribute that energy cyclically over geometric space and time in the manner described herein can be used to non-thermally ablate cancer cells while minimizing thermal damage to the target region and surrounding tissue. According to embodiments, the ratio of thermally damaged tissue area and ablation area is less than any of 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5% or 0.1%. This area is typically localized to the immediate area surrounding the electrodes, and in cases where electrode cooling is used, to the immediate area surrounding the cooling zone.

Thermal damage can be measured, for example, as an amount of white tissue coagulation, or an area of white tissue coagulation. Thermal damage can be measured by the amount of denaturization (e.g., protein denaturization or loss of extracellular matrix integrity). Higher levels of thermal damage include protein coagulation, tissue desiccation, and tissue vaporization. Thermal damage can also be measured indirectly, such as with one or more thermal sensor, such as measuring temperature of tissue, electrodes, and/or a region of tissue-electrode interface. According to one embodiment, one or more fiber optic thermal sensor is used to measure temperature. Thermal damage can also be measured by other means, such as by way of biomarkers (e.g. expression of heat shock proteins or heat shock protein genes). The area in which the onset of thermal damage occurs can be defined by Ω=2.3. Thermal damage is a metric of temperature and time and is typically quantitated using an Arrhenius equation or a dose calculation (CEM43). Typically, 43° C. is a commonly used value by those of skill in the art to illustrate the onset of thermal damage over 1 hour (albeit for skin burns).

The non-thermal ablation can occur by way of irreversible electroporation (IRE) and/or high frequency irreversible electroporation (HFIRE). While the following discussion is limited to IRE and HFIRE, it is contemplated that the principles of the invention can apply to other applications of delivering electrical energy to tissue, including electrical pulses capable of reversible electroporation, electrochemotherapy, electrogenetherapy, supraporation, or combinations thereof, such as by way of a DC current or AC current. Parameters for these applications are available in the literature.

Parameters which are effective for IRE and/or HFIRE can include the use of two or more electrodes or energized surfaces (e.g., two or more electrodes disposed in contact with one or the other tissue region, or two or more electrodes disposed in each or both), and from any number of electrodes or energized surfaces, such as 1, 2, 3, 4, 5, 6, 7, 8, 9, 10, 11, 12, 13, 14, 15, 16, 17, 18, 19, or 20 electrodes or energized surfaces and in any configuration relative to one another, such as being delivered by one or more pairs and/or delivered by one or more monopolar probe, one or more bipolar probe, or plate electrodes, such as three or more plate electrodes. The electrodes can also be positioned on flexible medical devices, such as catheters, endoscopes, bronchoscope tools, etc. The electrodes can be spaced apart in pairs such as electrodes spaced from 0 cm to 10 cm apart, such as from above 0 cm up to 10 cm apart, or from 0.2 cm to 9 cm, such as from 0.5 cm to 5 cm, or from 1 cm to 4 cm apart, or from 2 cm to 3 cm, or 1.5 cm, or any range in between any of these ranges or endpoints, including as endpoints any number encompassed thereby.

The electrodes can be needle electrodes, plate electrodes, blunt tip electrodes or combinations thereof. The electrodes can have a length (whether the length of the active tip of the electrode or the shaft of the probe) ranging from 1 cm to 30 cm, such as from 10 cm to 20 cm, or from 5 cm to 15 cm, and/or with a length of the active portion of the probe (e.g., energizable region) ranging from 0.5 cm to 10 cm, such as from 1 cm to 5 cm, or up to 3 cm or up to 4 cm, or any range in between any of these ranges or endpoints, including as endpoints any number encompassed thereby.

The delivering of pulses can be performed using a voltage for the plurality of electrical pulses of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or any range in between any of these ranges or endpoints, including as endpoints any number encompassed thereby.

The electrical pulses have a pulse length in the picosecond to second range, such as in the nanosecond to ms range, such as from 1 picosecond to 100 microseconds, or from 1 picosecond to 10 microseconds, or from 1 picosecond to 1 microsecond, or from at least 0.1 microsecond up to 1 second, or from 0.5 microseconds up to 10 microseconds, or up to 20 microseconds, or up to 50 microseconds, such as 15, 25, 30, 35, 40, 55, 60, 75, 80, 90, 110, or 200 microseconds, or any range in between any of these ranges or endpoints, including as endpoints any number encompassed thereby.

The plurality of electrical pulses can have a frequency in the range of 0 Hz to 100 MHZ, such as from above 0 Hz or 1 Hz up to 100 MHz. IRE pulses can have frequencies in the Hz range, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or any range in between any of these ranges or endpoints, including as endpoints any number encompassed thereby. HFIRE pulses can have frequencies in the kHz to tens or hundreds of kHz to several MHz range, such as from 1 kHz to 10 kHz, or from 2 kHz to 8 kHz, or from 3 kHz to 5 kHz, or from 4 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 75 kHz to 150 kHz, or from 100 kHz to 175 kHz, or from 200 kHz to 250 kHz, or from 225 kHz to 500 kHz, or from 250 kHz to 750 kHz, or from 500 kHz to 1 MHz, or any range in between any of these ranges or endpoints, including as endpoints any number encompassed thereby. HFIRE is described in U.S. Pat. Nos. 10,292,755 and 10,448,989, which are incorporated herein in their entireties.

The plurality of electrical pulses can have a waveform that is square, triangular, trapezoidal, exponential decay, sawtooth, sinusoidal, bipolar and/or alternating polarity.

According to one embodiment, one or more of the following parameters are employed for the delivering of the electrical pulses:
a) a number of cycles from one to ten (e.g., where one cycle comprises activating all possible electrode pair combinations at least once, or activating a select group of the electrode pair combinations, or sequentially activating all or less than all of the possible pair combinations);
b) one or more delays of 0 to 10 seconds each (within a pulse train and/or between activation of pairs and/or between cycles);
c) a number of pulses per cycle and/or pulses per pair of 10 to 200;
d) a total number of pulses of 100 to 5000; and/or
e) a total number of electrode pair combinations of 1 to 30.

### Systems

Embodiments of the invention include systems capable of performing one or more methods described herein, or one or more portions thereof. The systems can have therapeutic or diagnostic utilities or applications, or combinations thereof, according to various implementations. Therapeutic applications include ablating, such as substantial non-thermal ablating, one or more tumor or portion thereof. Diagnostic applications include determining the presence of thermal damage, an area or volume of thermal damage, or a probability of thermal damage.

An embodiment of an ablation system includes at least a first and second electrode, a voltage generator programmed to generate a plurality of electrical pulses between the first and second electrodes in a manner which causes non-thermal ablation of cells of one or more tissue, a memory, and a processor. The ablation system can include multiple electrodes such as an array of electrodes implemented with a support capable of positioning the electrodes of the array at predetermined distances from each other. The ablation system can also include one or more thermal sensor, such as a fiber optic thermal sensor, disposed on, within, or adjacent to the electrodes. The ablation system can also include one or more optical imaging system capable of measuring thermal damage, such as white tissue coagulation.

An embodiment includes an electrical energy-based system which includes an array of three or more electrodes and an electrical pulse generator in operable communication with the electrodes. The electrical pulse generator includes programming capable of distributing a total number of electrical pulses or bursts to a target tissue in a manner which reduces thermal damage and/or reduces thermal effects and/or minimizes potential for Joule heating and/or reduces delivery of electric current per unit time. The system's programming is capable of activating a plurality of pairs of electrodes in a manner which distributes an electric field at different spatial coordinates at different times. The programming is capable of applying a pulse train of a predetermined number of pulses or bursts to one pair of electrodes at a time during the activating. The programming is capable of distributing the total number of pulses over multiple pairs, wherein each pair is individually activated by a pulse train, and pairs are activated over multiple cycles such as according to Equation 1. The programming is capable of introducing one or more delays between one or more of the pulses, such as within one or more pulse train applied to each pair, and/or between the activation of one or more electrode pair, and/or between one or more of the cycles. In embodiments, the programming can be pre-programmed and/or a user/practitioner can input certain parameters, such as the number of pulses/bursts and/or number of cycles.

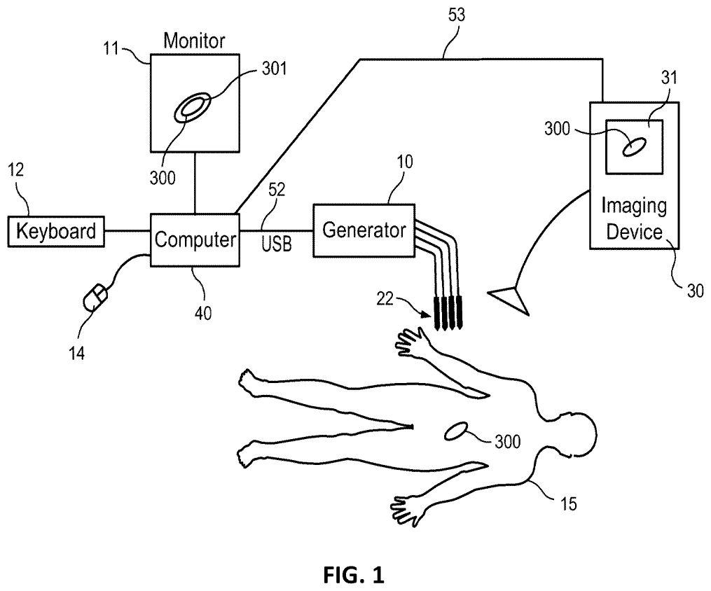

A system embodiment which is capable of executing any method described herein or portion thereof is illustrated in FIG. 1. One or more electrodes/probes 22 deliver therapeutic energy and are powered by a voltage pulse generator 10 that generates high voltage pulses as therapeutic energy such as pulses capable of ablating (e.g. irreversibly electroporating) the tissue cells. The voltage pulse generator 10 can include one or more and any number of receptacles for receiving up to a desired number of individual probes 22 which are adapted to be plugged into the respective receptacle.

Each probe 22 can include one or more monopolar electrode, one or more bipolar electrode having at least two electrodes (electrode conducting regions) separated by an insulating sleeve, multipolar electrodes having greater than two electrode surfaces separated by one or more insulating sleeves which can be energized simultaneously or at different times, plate electrodes with multiple surface electrodes, and/or a grounding pad. In one embodiment, if the probe includes a monopolar electrode, the amount of exposure of the active portion of the electrode can be adjusted by retracting or advancing an insulating sleeve relative to the electrode. See, for example, U.S. Pat. No. 7,344,533, which is incorporated by reference herein in its entirety. In other embodiments, such as when grounding pads are used, the energy can be distributed/alternated between them. For example, any number of grounding pads can be used to mitigate heat accumulating on any one particular grounding pad. In the embodiment shown, the probes 22 are monopolar electrodes. The generator 10 is connected to a treatment control computer 40 having input devices such as keyboard 12 and a pointing device 14, and an output device such as a display device 11 for viewing an image of a target treatment area such as a lesion 300 surrounded by a safety margin 301. The communications connections can be wired or wireless. The therapeutic energy delivery device 20 is used to treat a lesion 300 inside a patient 15. An imaging device 30 includes a monitor 31 for viewing the lesion 300 inside the patient 15 in real time. Examples of imaging devices 30 include ultrasonic, CT, MRI, PET, and fluoroscopic devices as are known in the art. The imaging device 30 can also be a camera capable of capturing digital images, microscopic images, and the like.

For purposes of this application, the terms “code”, “software”, “program”, “programming”, “application”, “software code”, “software module”, “module”, “program module”, and “software program” are used interchangeably to mean software instructions that are executable by a processor. The computer-executable instructions may be organized into routines, subroutines, procedures, objects, methods, functions, or any other organization of computer-executable instructions that is known or becomes known to a skilled artisan in light of this disclosure, where the computer-executable instructions are configured to direct a computer or other data processing device to perform one or more of the specified methods, processes and operations described herein. The computer-executable instructions may be written in any suitable programming language, non-limiting examples of which include C, C++, C #, Objective C, Swift, Ruby/Ruby on Rails, Visual Basic, Java, Python, Perl, PHP, MATLAB and JavaScript.

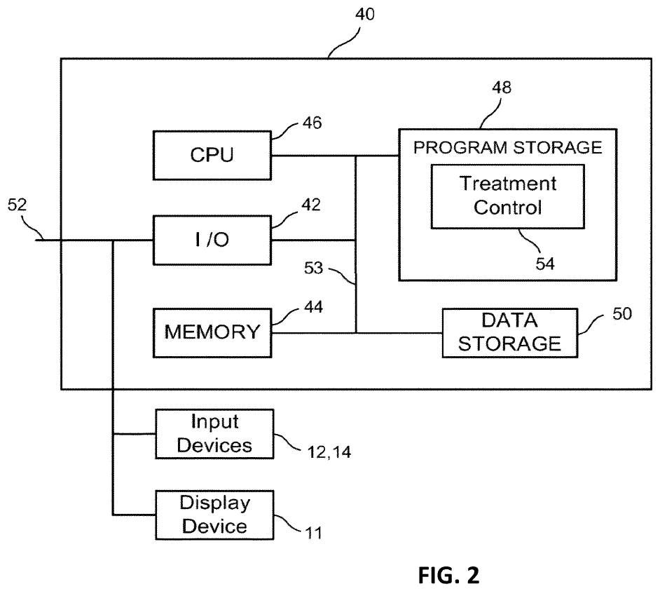

The “user” of the system can be a physician, a practitioner, or other medical professional. The treatment control module 54 (FIG. 2) executed by a processor outputs various data including text and graphical data to the monitor 11 associated with the generator 10.

Referring now to FIG. 2, the treatment control computer 40 of the present invention is connected to the communication link 52 through an I/O interface 42 such as a USB (universal serial bus) interface, which receives information from and sends information over the communication link 52 to the voltage generator 10. The computer 40 includes memory storage 44 such as RAM, processor (CPU) 46, program storage 48 such as ROM or EEPROM, and data storage 50, such as hard disk, all commonly connected to each other through a bus 53. As used herein, “memory storage”, “program storage”, and “data storage” are interchangeable with “non-transitory computer readable storage medium”. Program storage 48 stores, among others, computer software (treatment control module 54) or programming which assists a user/physician to plan for, execute, and review the results of a medical treatment procedure, and execute any of the methods described herein. The treatment control module 54, executed by the processor 46, assists a user to plan for a medical treatment procedure by enabling a user to more accurately position each of the probes 22 of the therapeutic energy delivery device 20 in relation to the lesion 300 in a way that will generate the most effective treatment zone. The treatment control module 54 can display the anticipated treatment zone based on the position of the probes and the treatment parameters.

Using any of the methods described herein, the treatment control module 54 can determine/administer a protocol which distributes a total number of pulses (chosen and inputted by a user, or calculated by the treatment planning module) of administered electrical energy to different pairs of the electrodes over a plurality of cycles in a manner which minimizes Joule heating and resulting thermal damage. The treatment control module 54 can also introduce delays in the pulsing protocol to minimize Joule heating, such as a delay within one or more pulse train applied to an electrode pair, and/or a delay between activation of one or more electrode pairs, and/or a delay between one or more cycles. Further, using any of the methods described herein, the treatment control module can alternatively allow a user to manually input a protocol, or any number of one or more parameters, which distributes a total number of pulses of administered electrical energy to different pairs of the electrodes over a plurality of cycles, and/or includes one or more delays between pulse administration. Based on the parameters entered by the user, the treatment control module can then calculate an area of anticipated thermal damage, an anticipated area of non-thermal ablation, and display a visualization of these areas to the user, as well as display an estimate of the ratio of anticipated thermal damage to non-thermal ablation, such as on the monitor or display device 11. The displays can be made available in real-time, for example, to make real-time temperature changes available on screen. Additionally, the treatment control module can modify protocols (whether determined manually by the user or determined by the treatment control module) based on heat received from the thermal sensors, or other feedback, such as imaging of thermal damage. The treatment control module can also display the progress of the treatment in real time and can display the results of the treatment procedure after it is completed. This information can be used to determine whether the treatment was successful and whether it is necessary to re-treat the patient.

The module 54 is also adapted to monitor and display the progress of the electroporation procedure and to determine a successful end point based on the electrical properties of the tissue prior to and during the treatment procedure as explained in more detail in U.S. Patent Application Publication No. 20190175248A1, which is incorporated by reference herein in its entirety. Being able to in real-time monitor and see the end point of the treatment procedure is a huge advantage over the current method in which the physician is performing the treatment essentially blindly without having any idea about whether the treatment is progressing or at what point the treatment procedure is finished.

The program storage 48 stores various electrical threshold values that are used to monitor the treatment procedure. When the programmed sequence of pulses has been delivered and the end point of the procedure has not been reached, the user interface portion of the control module 54 retrieves the recommended parameter changes from the database and presents them to the user through the display 11. The treatment control module 54 can also change the threshold values for determining the progress and the end point of the procedure based on initial treatment pulse parameters programmed by the user. A user can manually input the various thresholds for different tissue types or the system can have these thresholds stored electronically.

Alternatively, the treatment control module 54 can also automatically derive or adjust the threshold values for determining the progress and the end point of the procedure based on test signals (e.g., AC test signals) that are applied and determining electrical properties of the cells such as impedance values or current values. The control module 54 may then store the changed threshold values in the program storage 48 for later use as the new criteria for comparison.

Further, AC intra-treatment test signals may continue to be delivered in addition to the comparative DC intra-treatment test signals. By tracking the change in impedance for the AC-signal, the treatment control module 54 determines and factors out the effects on impedance occurring due to temperature rise. This enables more accurately tracking changes in the real-part of the impedance by reflecting changes encountered solely due to persistent electroporated cells. A more detailed discussion of the control module 54 is made in U.S. Patent Application Publication No. 20190175248A1, which is incorporated by reference herein in its entirety.

Any of the software program modules in the program storage 48 and data from the data storage 50 can be transferred to the memory 44 as needed and is executed by the CPU 46.

In one embodiment, the computer 40 is built into the voltage generator 10. In another embodiment, the computer 40 is a separate unit which is connected to the voltage generator through the communications link 52. The communication link 52 can be, for example, a USB link.

In one embodiment, the imaging device 30 is a stand-alone device which is not connected to the computer 40. In the embodiment as shown in FIG. 1, the computer 40 is connected to the imaging device 30 through a communications link 53. As shown, the communication link 53 is a USB link. In this embodiment, the lesion image generated by the imaging device 30 can be directly displayed on the monitor 11 of the computer running the treatment control module 54. The image data from the imaging device 30 can also be used to capture information during and/or after treatment of the lesion 300, such as an area of ablation and/or an area of thermal damage.

It should be noted that the software can be used independently of the generator 10. For example, the user can plan the treatment in a different computer as will be explained below and then save the treatment parameters to an external memory device, such as a USB flash drive (not shown). The data from the memory device relating to the treatment parameters can then be downloaded into the computer 40 to be used with the generator 10 for treatment.

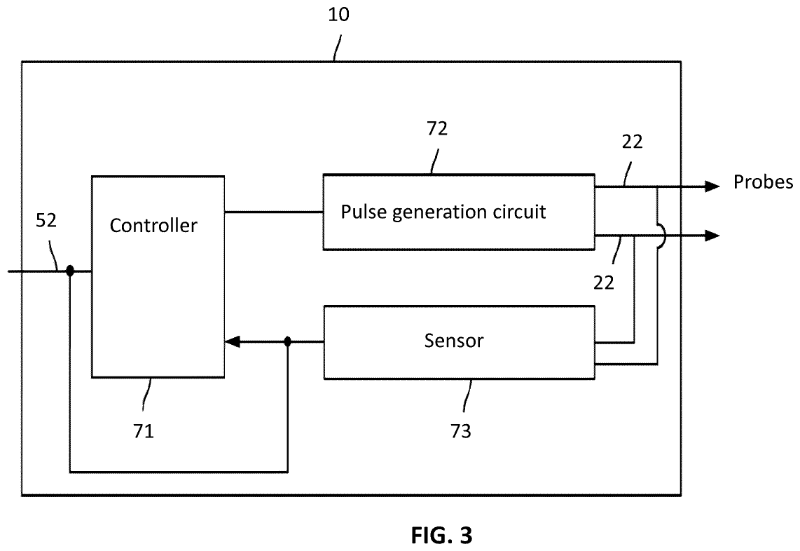

FIG. 3 is a functional block diagram of a pulse generator 10 shown in FIG. 1. FIG. 2 illustrates one embodiment of a circuitry to monitor the progress of and determine an end point of the treatment procedure. A USB connection 52 carries instructions from the user computer 40 to a controller 71. The controller 71 can be a computer similar to the computer 40 as shown in FIG. 2. The controller 71 can include a processor, ASIC (application-specific integrated circuit), microcontroller or wired logic. The controller 71 then sends the instructions to a pulse generation circuit 72. The pulse generation circuit 72 generates the pulses and sends electrical energy to the probes. For clarity, only one pair of probes/electrodes is shown. However, the generator 10 can accommodate any number of probes/electrodes such as 1 probe, 2 probes, 3 probes, 4 probes, 5 probes, 6 probes, 7 probes, 8 probes, 9 probes, 10 probes, 11 probes, 12 probes, 13 probes, 14 probes, 15 probes, 16 probes, 17 probes, 18 probes, 19 probes, 20 probes, or more. In the embodiment shown, the pulses are applied one pair of electrodes at a time, and then switched to another pair. The pulse generation circuit 72 includes a switch, preferably an electronic switch that switches the probe pairs based on the instructions received from controller 71.

A sensor 73 can sense the current and voltage between each pair of the probes in real time and communicate such information to the controller 71, which in turn, communicates the information to the computer 40. Although the treatment control module 54 houses the software code for monitoring the treatment procedure, it may be beneficial for the controller 71 to store such module as the speed of monitoring can be important in some cases.

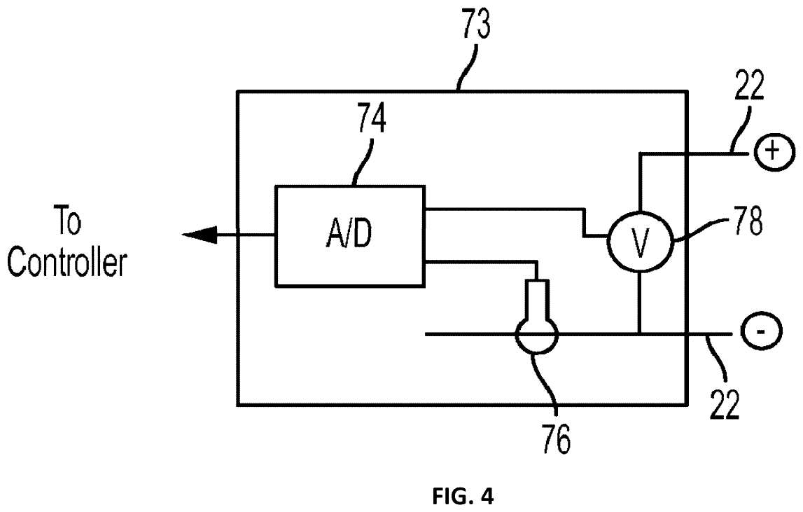

FIG. 4 is a functional block diagram of a sensor 73 of FIG. 3. The sensor 73 includes a voltage, current, or impedance sensor 78 connected across a pair of electrodes 22 and a current sensor or output monitor 76 connected to a negative electrode (return conduit) in the pair of electrodes. Although FIGS. 3-4 show two electrodes from two wires 22, there may be multiple electrodes between the two wires 22. The sensed values are continuously received and digitized by an A/D converter 74 and transmitted to the controller 71. Preferably, the A/D converter 74 can sample the sensed values at a very fast rate and preferably at a rate of at least 100 MHZ (100 million samples per second) for the control module 54 to be able to accurately assess the complex impedance of test signals which may be an AC signal at a relatively high frequency.

The current sensor 76 can be a Hall effect sensor/probe which is positioned around an electrode so as to measure the electric current without directly interfering with the pulse signal. Typically, the current sensor 76 is placed on the negative signal connection of the electrode pair. If the electrode pairs are switched, then only one current sensor connected at the input side of the switch is needed. Otherwise, if there are 3 pairs of electrodes, for example, and all are firing at the same time, there will be 3 current sensors so as to measure the electric current of each pair separately. In that case, the current from the three sensors will need to be added. Such embodiments are helpful in circumstances where the practitioner desires to stay below a certain current threshold. The inventive activation protocols can also be used to minimize the amount of current delivered to the target region during the treatment. For example, rotating or distributing the application of energy in the treatment zone can keep the amount of current delivered at a minimum. The amount of current being delivered can be monitored, such as using sensor(s), and the activation protocol adjusted accordingly to avoid current rising above a desired threshold. For example, a practitioner monitoring the current could adjust the protocol to skip a cycle or reduce the number of pulses/bursts being applied if the current readings from the sensor indicate the amount of current is getting too high.

The voltage sensor 78 can be a conventional voltage divider, comprised of two serially connected resistors, that measures a voltage drop across a known resistance value. The voltage sensor 78 uses resistors which are of much higher resistance than the tissue (kΩ-MΩ, versus tissue, which is hundreds of Ω), and thus induces negligible effect on the strength of the pulses delivered to the tissue. A correction factor is calculated for the divider circuit based on the resistances of the two resistors in the voltage divider circuit and the resistance of the load (tissue resistance) to determine the true delivered voltage to the tissue based on the measured voltage drop across the resistor.

The software program modules in the program storage 48 and data from the data storage 50 can be configured so that one or more treatment parameter such as current can be inputted, stored, and/or displayed to the user/physician on the display device 11. The one or more treatment parameter can be stored in the data storage 50, and values such as the absolute values or average values of the one or more treatment parameter or relative values, such as the amount of change in the one or more treatment parameter over time, can be inputted, stored, and/or displayed by the user/physician. In one embodiment, the treatment parameter values are stored as thresholds.

The software program modules in the program storage 48 can include programming configured to determine an average value or a change in value of a non-thermal ablation treatment parameter measured in real time during treatment. The programming can input measured values of a treatment parameter during real time monitoring such as current, and calculate an average value over time, or a change in value, such as from a baseline. The baseline can be established at various time points before or during treatment.

Now having described the embodiments of the present disclosure, in general, the following Example describes some additional embodiments of the present disclosure. While embodiments of the present disclosure are described in connection with the following example and the corresponding text and figures, there is no intent to limit embodiments of the present disclosure to this description. On the contrary, the intent is to cover all alternatives, modifications, and equivalents included within the spirit and scope of the principles of the present disclosure.

### EXAMPLE

Purpose: To evaluate the effects of various pulsing paradigms, on the irreversible electroporation (IRE) lesion, induced electric current, and temperature changes using a perfused porcine liver model.

Materials and Methods: A 4-monopolar electrode array delivered IRE therapy varying the pulse length and inter-pulse delay to six porcine mechanically perfused livers. Pulse paradigms included six forms of cycled pulsing schemes and the conventional pulsing scheme. Finite element models provided further insight into the effects of cycled pulsing on the temperature and thermal injury distribution.

Results: “Single pulse cycle with no interpulse delay” deposited maximum average energy (2.34±0.35 kJ) and produced the largest ratio of thermally damaged tissue area and IRE ablation area from all other pulse schemes (18.22%±8.11, p<0.0001 all pairwise comparisons). These compared favorably to the conventional algorithm (2.09±0.37 kJ, 3.49%±2.20, p<0.0001, all comparisons). Though no statistical significance was found between groups, the “5 pulse cycle, 0 s delay” pulse paradigm produced the largest average IRE ablation cross sectional area (11.81±1.97 cm2), while conventional paradigm yielded an average of 8.90±0.91 cm2. Finite element modeling indicated “10 pulse cycle, 10 s delay” generates the least thermal tissue damage and “1 pulse cycle, 0 s delay” pulse cycle sequence the most (0.47 vs. 3.76 cm2), over a lengthier treatment time (16.5 vs. 6.67 minutes).

Conclusions: Subdividing IRE pulses and adding delays throughout the treatment can reduce white tissue coagulation and electric current, while maintaining IRE treatment sizes. For example, see “10 pc/5 s delay” and “10 pc/10 s delay” protocols with either conventional or enhanced EPAP, as shown in FIGS. 12A-C.

### Introduction

Irreversible Electroporation (IRE) is an energy directed therapeutic used to treat patients with unresectable tumors. This focal ablation technique relies on the application of brief, high-amplitude, pulsed electric fields between two or more needle applicators/electrodes to increase the transmembrane potential of a cell above a critical threshold, destabilize the lipid bilayer of the cell membrane, increase the cell-tissue permeability, and ultimately kill cells within the targeted region. The overall ablation volume is contingent on the underlying tissue properties and the various energy delivery parameters. Energy delivery can be partially defined as a function of the physical delivery system (the electrode geometry, the number of electrodes utilized, and electrode spacing), as well as the systematic electrical pulse parameters (voltage amplitude, pulse frequency, and pulse repetition). The proper tuning of these electrode properties and pulse parameters can enable a targeted, non-thermal treatment of local and inoperable tumors. Consequently, this minimally invasive technique can be used to target malignancies enveloping critical structures (blood vessels, major nerves, etc.) and is less influenced by the convective effects of local blood perfusion (heat sink effect) in comparison to thermal ablative technologies (radiofrequency ablation, and microwave ablation, etc.) (see B. E. Bulvik, M. Ahmed, A. V Andriyanov, and S. N. Goldberg, “Irreversible Electroporation versus Radiofrequency Ablation : A Comparison of Local and Systemic,” vol. 280, no. 2, 2016).

Treatment of large tumors benefit from higher voltages which in turn cause moderate temperature and current variations; the latter has been linked to overall treatment outcomes (see E. Ben-David et al., “Irreversible Electroporation: Treatment Effect Is Susceptible,” Radiology, vol. 269, no. 3, 2013; B. E. Bulvik, M. Ahmed, A. V Andriyanov, and S. N. Goldberg, “Irreversible Electroporation versus Radiofrequency Ablation: A Comparison of Local and Systemic,” vol. 280, no. 2, 2016. Studies have shown that while a higher delivered electrical energy can be associated with larger treatment zones, it also causes an increase in white tissue coagulation due to Joule heating effects. Some thermal mitigation strategies have been explored, including methods to absorb and remove heat via internal perfusion and the use of phase change materials within the electrode/applicator core. However, these methods may increase operational complexity and/or require thicker electrodes. In many instances, clinicians performing IRE treatments will also rely on increasing the number of monopolar IRE applicators to three or more to encompass a large or irregularly shaped tumor. On these occasions, conventional IRE generators would deliver the entirety of the set number of pulses for a given electrode pair before proceeding to the next electrode pair, until all of the desired electrode pair combinations have been activated making this procedure time consuming. Moreover, while the addition of electrodes may be useful in generating a larger ablation, the existing method of pulse delivery has not been engineered to moderate thermal tissue damage.

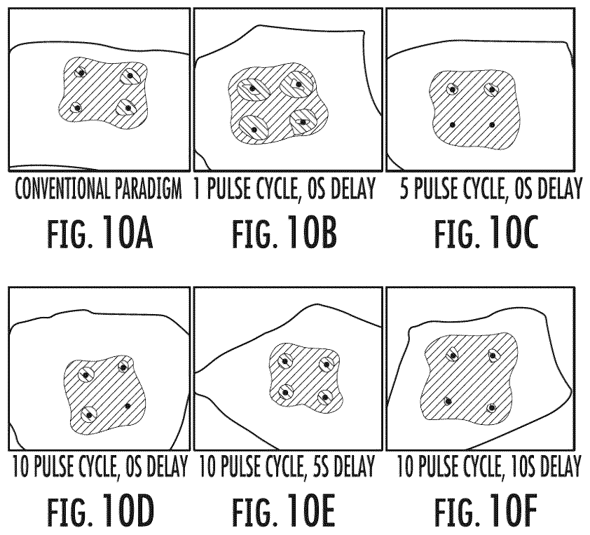

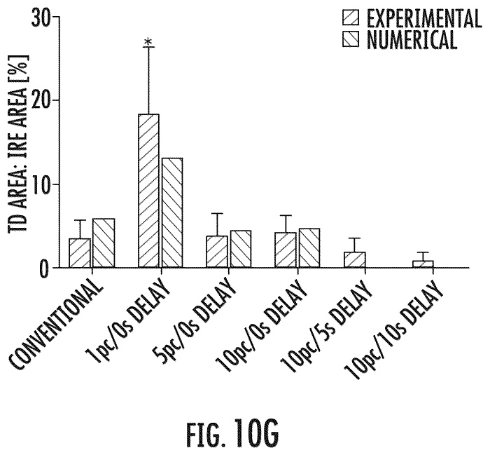

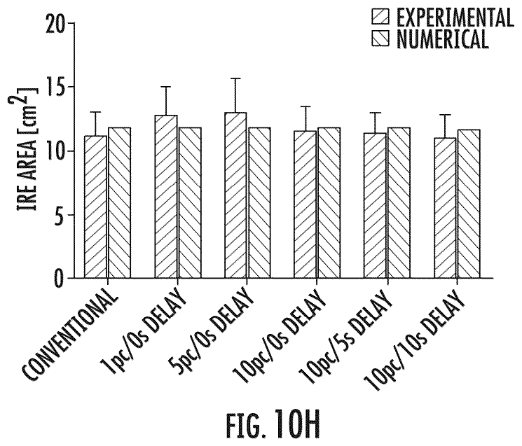

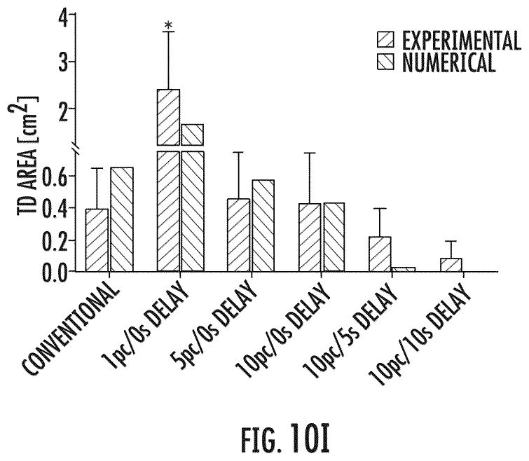

The inventors have investigated the effects of various cycled pulsing paradigms in comparison to conventional pulsing schemes via a multi-electrode IRE therapy (4-electrode configuration) on the IRE treatment zone size, deployed electrical current, temperature changes, and treatment time. The inventors have discovered that subdividing the number of pulses delivered per electrode pair and adding delays throughout the treatment are techniques that can be used to reduce tissue temperatures, electric current, and the overall thermal damage while maintaining IRE treatment zone sizes. For example, as shown in FIG. 10I, the “10 pulse cycle, 10 s delay” pulse cycle sequence illustrated a 99.3% reduction of thermal damage area in comparison to the conventional pulse scheme, and each of the multi-cycle pulsing paradigms illustrated less area thermal damage on average than the single-cycle scheme (“1 pulse cycle, 0 s delay” and the conventional pulse scheme). Additionally, as shown in FIGS. 12A-B, the incorporation of cycled pulsing, with an enhanced EPAP could reduce the surface area and volumetric thermal damage as much as 13.6% in comparison to the conventional pulse paradigm, while maintaining the effective electric field thresholds within 2.1%. Further, an adjustment to the order in which each electrode pair is activated during treatment was also explored in an attempt to further minimize the potential for Joule heating.

### Materials and Methods

### Electroporation Pulse Delivery

All IRE treatments were performed in a perfused organ model (see S. Bhonsle et al., “Characterization of Irreversible Electroporation Ablation with a Validated Perfused Organ Model,” J. Vasc. Interv. Radiol., vol. 27, no. 12, pp. 1913-1922.e2, 2016; and T. J. OBrien et al., “Effects of internal electrode cooling on irreversible electroporation using a perfused organ model,” Int. J. Hyperth., vol. 35, no. 1, pp. 44-55, 2018). A total of six livers were used for this study with 6-8 treatments delivered per liver using a 4-electrode configuration electrode array. Five cycled pulsing variations were performed (Table 2) and evaluated in comparison to a conventional algorithm. As shown in FIGS. 5A-C, a 4-electrode array was used to deliver the various pulse protocols, including a conventional pulse protocol (FIG. 5A) using a conventional electrode pair activation pattern (EPAP), a cycled pulsing protocol (FIG. 5B) also using a conventional EPAP (and the same EPAP as in FIG. 5A), and a cycled pulsing protocol (FIG. 5C) using an enhanced EPAP. As shown in each of FIGS. 5A-C, the activated electrode is shown encircled in bold and the arrow denotes the flow of energy toward to other electrode in the pair being activated. The pairs of activated electrodes are shown encircled with an oval.

Conventional pulsing protocols typically deposit the total number of pulses/bursts desired across one electrode pair before moving to the next electrode pair, as illustrated within FIG. 5A where 100 pulses are deposited per electrode pair. This pulsing algorithm includes a 3.5 s delay per every 10 pulses to recharge the capacitor bank, which provides some thermal mitigation.

By comparison, the cycled pulsing schemes investigated differ by partitioning of the pulse-train into subsets of pulses (30, 60, 120, etc.) and cycling between active electrode pairs until the desired final number of pulses is achieved per electrode pair (FIG. 5B). Thus, the “5 pulse cycle, 0 s delay” scheme applies 20 pulses per electrode pair (6 electrode pairs), yielding a total of 120 pulses per “cycle”. This procedure is repeated until a total of 600 pulses are applied (i.e. a total of 5 cycles). While there is no delay between activated electrode pair pulsing sequence associated with this “5 pulse cycle, 0 s delay” method, the addition of a delay between each cycle was also investigated. In embodiments, delays can take the form or any one or more of inter-pulse delays (one or more delay between pulses), inter-burst delays (one or more delay between bursts of pulses), and/or inter-cycle delays (one or more delay between cycles of pulses or bursts being applied). Lastly, as thermal damage is typically associated with temperature and on time, the order in which electrode pairs activate was examined to minimize consecutive electrode activation and thus allow the tissue more time to rest between activations and reduce the amount of unwanted thermal damage to the tissue. FIG. 5C shows one such enhanced electrode pair activation pattern.

More particularly, in embodiments such as the cycled pulsing protocol of FIG. 5B, a conventional electrode pair activation pattern (EPAP) is used. Typically, in such a protocol the electrode pairs with the largest distance between them (e.g., the diagonal electrodes) are activated first. Then since the remaining electrode pair combinations each have the same distance of separation between the electrodes in each pair (e.g., in a 4-electrode configuration), any of the remaining electrode pairs are activated sequentially/consecutively until all pairs have been activated. With such a protocol, a single electrode can be activated three times in a row.

Cycled pulsing embodiments that use an enhanced electrode pair activation protocol (EPAP) as in FIG. 5C, typically can start out similarly to that of the beginning of the protocol in FIG. 5B, where (1) the electrodes with the largest separation distance (e.g., the diagonal electrodes) are activated first, (2) and any of the remaining electrode pairs can be activated next, but differing from the FIG. 5B protocol in that (3) each of the remaining electrode pairs is activated in an order such that the electrodes of the electrode pair were not activated in the last activation. For example, activating a pair of electrodes opposite the last pair of electrodes activated avoids sequential/consecutive activation of an electrode. This “enhanced” EPAP results in allowing the tissue more time to rest between electrode pair activations. As shown in FIG. 12B, typically the amount of tissue damage for the enhanced EPAP protocols was less than the amount associated with the conventional EPAP protocols.

TABLE 2
Summary of the treatment variations of cycled pulse sequencing. Six different pulsing paradigms
were compared. Conventional pulsing sequences typically incorporate a 3.5 second delay per every
10 pulses/bursts (due to capacitor charging), while all other pulsing paradigms implement their
delays between cycles. Pulse delivery was iterated between four monopolar electrodes equally
spaced apart by 2 cm, with an electrode exposure of 1.5 cm. The IRE generator was set to deliver
600 pulses at a rate of 90 pulses per minute and a constant voltage of 2700 V.

| | | | | No. | Electrode | Electrode | Pulse | Pulse |
|---|---|---|---|---|---|---|---|---|
| | No. | Delay | No. Pulses | Total | Separation | Exposure | Amplitude | Width |
| Parameter Set | Cycles | [s] | per Cycle | Pulses | [cm] | [cm] | [V] | [μs] |
| Conventional | 1 | 3.5 per | 100 | 600 | 2 | 1.5 | 2700 | 100 |
| Paradigm | | 10 | | | | | | |
| | | pulses | | | | | | |
| 1 pulse cycle, | 1 | 0 | 100 | 600 | 2 | 1.5 | 2700 | 100 |
| 0 s delay | | | | | | | | |
| 5 pulse cycle, | 5 | 0 | 20 | 600 | 2 | 1.5 | 2700 | 100 |
| 0 s delay | | | | | | | | |
| 10 pulse cycle, | 10 | 0 | 10 | 600 | 2 | 1.5 | 2700 | 100 |
| 0 s delay | | | | | | | | |
| 10 pulse cycle, | 10 | 5 | 10 | 600 | 2 | 1.5 | 2700 | 100 |
| 5 s delay | | | | | | | | |
| 10 pulse cycle, | 10 | 10 | 10 | 600 | 2 | 1.5 | 2700 | 100 |
| 10 s delay | | | | | | | | |

### Perfused Whole Organ Tissue Testing

### Organ Preparation

All experimental IRE procedures are performed on a previously validated perfused organ model (see S. Bhonsle et al., “Characterization of Irreversible Electroporation Ablation with a Validated Perfused Organ Model,” J. Vasc. Interv. Radiol., vol. 27, no. 12, pp. 1913-1922.e2, 2016). Porcine livers were excised at a local abattoir and immediately fit with Luer-Lock connectors at the major ports of the specimen for a constant pressure flush (˜90 mmHg) with three liters of modified phosphate buffer solution (conductivity of ˜0.8 S/m) to mitigate thrombosis and improve overall tissue perfusion. The tissue was then transported via static cold storage (SCS) until anastomosed to the organ preservation system (˜120 minutes on SCS). The perfusate temperature on the preservation system was set and maintained at 30° C. After IRE treatments were delivered, organs were maintained on the system for an additional 2 hours at hypothermic conditions, 4° C., and prior to sectioning/staining. A total of six livers were used, with 6-8 treatments performed per liver (total N=42). All animals were euthanized and handled in strict accordance with good animal practice as defined by the relevant national and local animal welfare bodies, and approved by Virginia Tech.

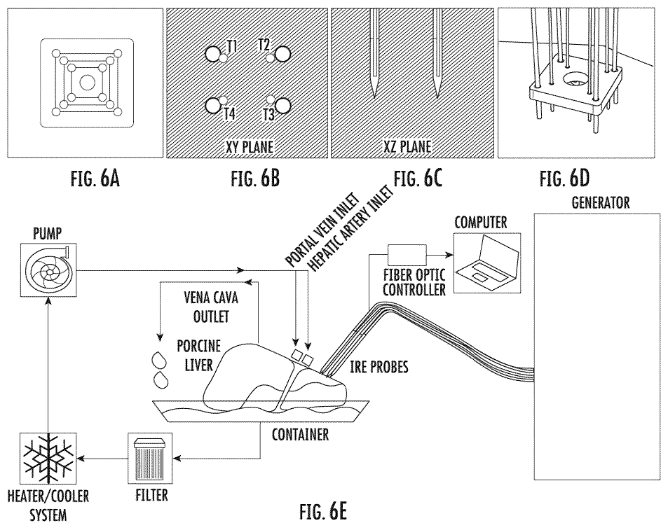

To ensure that all electrodes were equidistant for each treatment performed during these experiments, a custom built “support block” was designed and laser-cut from acrylic material (CAD diagram shown in FIG. 6A). FIG. 6D illustrates the monopolar support block for the 4-monopolar electrode configuration, FIGS. 6B and 6C show fiber optic sensor placement relative to the electrode array, and FIG. 6E shows a detailed schematic of the machine perfused liver model.

### Morphologic Analysis

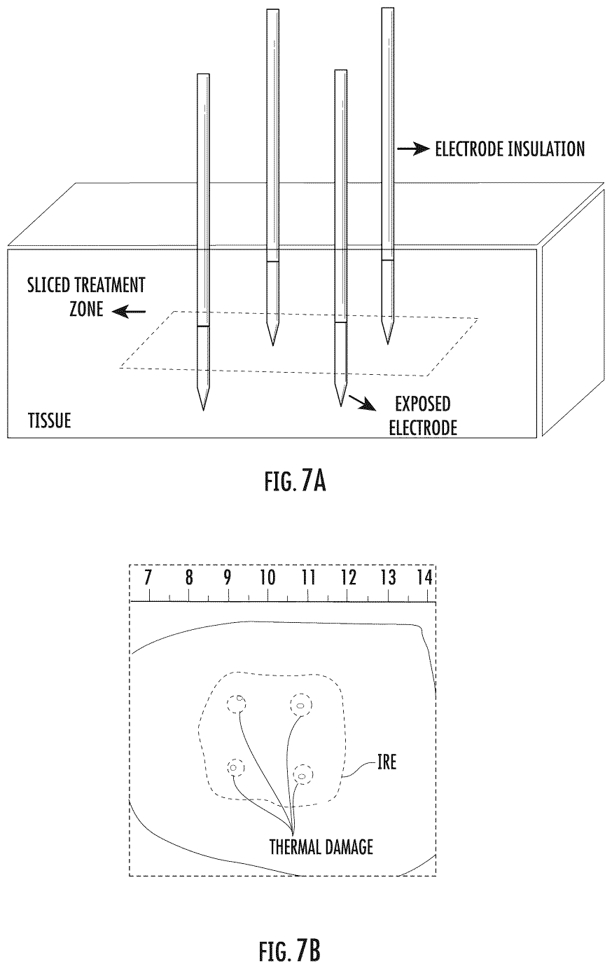

Sectioning was performed 2 hours after the last ablation performed to allow for IRE changes to manifest (see E. W. Lee, C. Chen, V. E. Prieto, S. M. Dry, C. T. Loh, and S. T. Kee, “Advanced Hepatic Ablation Technique for Creating Complete Cell Death: Irreversible Electroporation,” Radiology, vol. 255, no. 2, pp. 426-433, 2010). The total perfusion time was no more than 6 hrs. The tissue was sectioned and sliced through the mid-plane of the treatment regions for gross evaluation. These sections were stained with triphenyl tetrazolium chloride (TTC) (MP Biomedicals, LLC Santa Ana, California) in phosphate buffered saline (15 g/L) for 5-10 minutes without light exposure. Following staining, the treatment zones were photographed and measured along the long and short axis to determine the overall area of the treatment region. Samples were fixed in formalin overnight and measured once more 12 hours later. Prior studies have shown no statistical difference in ablation size when comparing the long and short axis measurements pre-and post-formalin (see M. Bonakdar, E. L. Latouche, R. L. Mahajan, and R. V. Davalos, “The feasibility of a smart surgical probe for verification of IRE treatments using electrical impedance spectroscopy,” IEEE Trans. Biomed. Eng., vol. 62, no. 11, pp. 2674-2684, 2015; and E. L. Latouche, M. B. Sano, M. F. Lorenzo, R. V. Davalos, and R. C. G. Martin, “Irreversible electroporation for the ablation of pancreatic malignancies: A patient-specific methodology,” J. Surg. Oncol., vol. 115, no. 6, pp. 711-717, 2017). Thus, the post formalin measurements were used to represent ablation size throughout the Example. ImageJ software (NIH, Bethesda, MD) was used to analyze and measure the thermally damaged regions and IRE ablated regions of each tissue sample. Briefly, threshold techniques were utilized to identify the thermally damaged tissue (discolored tissue regions) and IRE ablation (pale discolored tissue regions). Then, the average thermally damaged area and average IRE ablation area were expressed as percentage ratio of thermal damage to IRE ablation. FIGS. 7A-B illustrate the sectioning and ablation measurement process. It should be noted the calculated IRE ablation area also includes the thermally damaged area (area of white tissue coagulation); this combination was purposely included to calculate the percentage of thermal damage in proportion to the entire ablation, both nonthermal ablation and thermally damaged area.

### Thermal Measurements

Fiber optic thermal sensors (STB, Luxtron m3300, LumaSense, Santa Clara, CA, USA) were adhered to the surface, and positioned at the midpoint of each exposed electrode. The tissue temperature was measured during treatment at a rate of 0.5 samples per second. Temperature data was plotted over time to illustrate the thermal differences between each pulsing paradigm investigated. Further, the collected thermal data was utilized to validate the numerical models. FIGS. 6B and 6C specify the location of each fiber optic temperature sensor in relation to the electrodes.

### Current Measurements

The system generated .xml data files containing the treatment protocol and procedure, voltage, current, and resistance information. All data were imported and read using an in-house MATLAB program for analysis of the measured current. Similar data (voltage, current and resistance) were generated and saved for all cycled pulsing paradigms. The total energy delivered to the tissue was calculated via Equation 2:

$$Energy [ kJ ] = \frac{[ {V}_{avg} * {I}_{avg} ] * ( \#Pulses ) * ( 100 \times 1 {0}^{- 6} )}{1000} \qquad [ 2 ]$$

Where the average voltage, V_{avg}, and average current, I_{avg}, are defined as the area under the curve divided by the treatment length.

### Numerical Modeling of Varying Cycled Pulsing Paradigms

Numerical modeling was performed in parallel with experimental procedures to explore the effects of various cycled pulsing paradigms for multi-electrode IRE treatments in ex vivo porcine liver tissue. Previously measured experimental voltage and current data were employed to visualize electroporation-induced changes in electrical conductivity, thereby solving for more representative electric field distributions and applied electric currents. The finite element model was developed using COMSOL Multiphysics v5.4a (COMSOL Inc., Stockholm, Sweden). The domains consisted of liver tissue modeled as a 12×12×8 cm ellipsoid and four monopolar electrodes modeled as cylinders with height, diameter, and spacing of 1.5 cm, 1 mm, and 2 cm, respectively. All numerical model material properties are displayed in Table 3.

TABLE 3
Material properties used within the numerical model.

| Material | Parameter | Value | Units | Ref. |
|---|---|---|---|---|
| Liver | ρ, Density | 1079 | [kg/m³] | P. Hasgall et al. |
| | cₚ, Heat Capacity | 3540 | [J/kg/K] | P. Hasgall et al. |
| | k, Thermal | 0.52 | [W/m/K] | P. Hasgall et at. |
| | Conductivity | | | |
| | α, Thermal | 2 | [%/° C.] | P. Hasgall et at. |
| | Coefficient of | | | |
| | Conductivity | | | |
| | ω_{b}, Perfusion | 3.575 × 10⁻³ | [1/s] | P. Hasgall et al. |
| | σ, Electrical | 0.67 | [S/m] | P. Hasgall et al. |
| | Conductivity | | | |
| | Eₒ, Reversible | 460 | [V/cm] | D. Haemmerich |
| | Field Threshold | | | and B. J. Wood |
| | E₁, Irreversible | 700 | [V/cm] | D. Haemmerich |
| | Field Threshold | | | and B. J. Wood |
| Electrode | ρ, Density | 7900 | [kg/m³] | P. Hasgall et at. |
| | cₚ, Heat Capacity | 500 | [J/kg/K] | P. Hasgall et at. |
| | k, Thermal | 15 | [W/m/K] | P. Hasgall et al. |
| | Conductivity | | | |
| | σ, Electrical | 2.22 × 10⁶ | [S/m] | P. Hasgall et al. |
| | Conductivity | | | |

The final mesh consisted of 187,418 tetrahedral elements with a maximum of 2,264,706 degrees of freedom. The electric potential distribution was solved using the Laplace equation (Equation 3), and taking the gradient of the electric potential (Equation 4):

$$- ∇ \cdot ( σ ∇ ϕ ) = 0 \qquad [ 3 ]$$

$$\overline{E} = - ∇ Φ \qquad [ 4 ]$$

where σ represents the electrical conductivity, ϕ is the electric potential, and Ē is the electric field. Considering a four-electrode configuration, and assuming that all six electrode pair combinations were used within the treatment, a total of six current modules were required to solve for the effective electric field distribution, each of which had imposed boundary conditions of ϕ=V and ϕ=0. All external boundaries were set as electrically insulating with the boundary condition

$$\frac{\partial ϕ}{\partial n} = 0 .$$

Prior studies nave shown tissue electroporation results in an increase in bulk tissue electrical conductivity. This effect is captured in the model by using a sigmoidal curve:

$$σ ( \vert \overline{E} \vert ) = {σ}_{0} + \frac{{σ}_{f} - {σ}_{0}}{1 + D \cdot {e}^{\frac{- ( \vert \overline{E} \vert - A )}{B}}} \qquad [ 5 ]$$

where σ₀ is the initial non-electroporated conductivity, σ_{f} is the peak electroporated conductivity, |Ē| is the magnitude of the electric field at any given position, and D, B, and A are fitting terms. In the context of the perfused organ model, the empirically determined values for σ₀, σ_{f}, D, B, and A are 0.12 S/m, 0.42 S/m, 10, 30 V/cm, and 580 V/cm, respectively. Due to the increase ionic mobility during therapy, the electrical conductivity of biological tissue increases with temperature, and is incorporated within the model via the electrical conductivity temperature coefficient, α:

$$σ ( \vert \overline{E} \vert , T ) = σ ( \vert \overline{E} \vert ) \cdot [ 1 + α \cdot ( T - {T}_{0} ) ] \qquad [ 6 ]$$

For direct current (DC) and low-to-medium frequencies (30 kHz-1 GHz), α is a positive coefficient taking on values between 0-4%/° C. Here, the α coefficient was defined as 2%/° C.

Temperature profiles are simulated using Pennes' bioheat equation that models conductive heat transfer and includes effects of heat dissipation due to a distributed blood perfusion term. The bioheat equation was modified to incorporate IRE Joule heating effects through the addition of a Joule heating term. To reduce computational time, the energy delivered during one pulse (100 μs) was averaged over the pulsing period (1 s) by scaling this Joule heating term to the duty cycle of the pulse. Thus, discrete pulses can now be defined as a continuous heat source without complex modifications to the numerical time stepping.

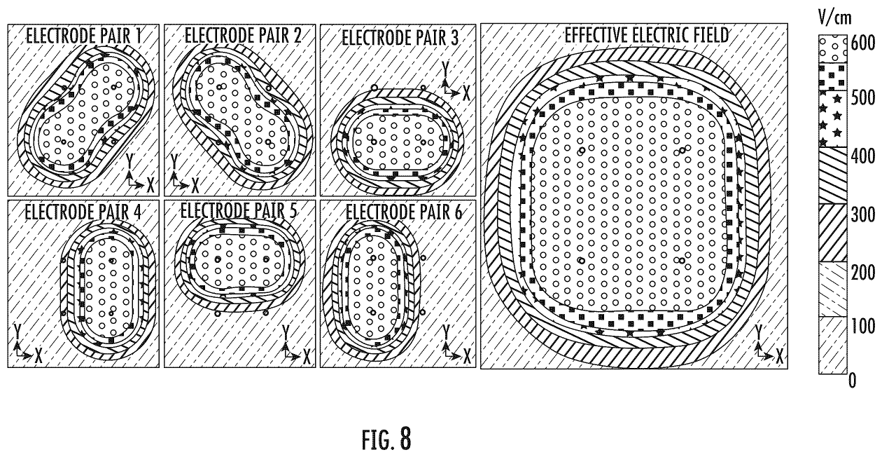

Lastly, the cycled pulsing schemes are implemented by combining the duty cycle approach with time dependent “thermal envelopes”. These thermal envelopes are implemented analytically where a defined coefficient χ is used to simulate the on (χ=1) and off (χ=0) period of the Joule Heating term. Since the Joule Heating term is a continuous source, these “thermal envelopes” enable the numerical model to mimic cyclic pulsing across various electrode pairs, with Joule Heating occurring only across the immediately energized electrode pair. This resulted in the following equation:

$$ρ {c}_{p} \frac{\partial T}{\partial t} = ∇ \cdot ( k ∇ T ) - {ω}_{b} {ρ}_{b} {c}_{b} ( T - {T}_{b} ) + \frac{σ \cdot {\vert \overline{E} \vert}^{2} \cdot p}{τ} \cdot χ \qquad [ 7 ]$$

where ρ describes the density of tissue, cₚ is the specific heat of tissue, κ is the thermal conductivity, ω_{b} is the perfusion, ρ_{b} describes the density of blood, c_{b} is the specific heat of blood, T_{b} is the arterial blood temperature, σ represents the electrical conductivity, p is the period per pulse, and τ represents the on-time per pulse. FIG. 8 illustrates the numerically calculated electric field distributions for each individual electrode pair and the cumulative or effective electric field using a conventional pulsing paradigm for a four electrode configuration. The area was calculated for tissue that exceeded electric field thresholds of 400, 600 800, and 1000 V/cm and both the conventional and enhanced EPAPs using a surface integration taken across the plane at the midpoint of the electrodes within the 3D model.

The accumulation of tissue thermal injury tissue due to pulsing was quantified using a thermal damage function:

$$Ω ( t ) = {\int }_{0}^{t} ζ \cdot {e}^{- \frac{{E}_{a}}{R \cdot T ( t )}} dt \qquad [ 8 ]$$

where ζ is the frequency factor (7.39×10³⁹ s⁻¹), Eₐ is the activation energy (2.577×10⁵ J/mol), R is the universal gas constant, T is the absolute temperature in Kelvin, and Ω, is the total injury due to thermal insult. A value of, Ω=2.3, was used to define the minimum conditions to obtain irreversible hepatic white tissue coagulation.

The total treatment time was calculated using the formula displayed in Equation 9:

$$Total Treatment Time [ min ] = \frac{[ ( \frac{{P}_{\#}}{{C}_{\#} \cdot f} ) + ( D ) ] ( {ε}_{p} ) ( {C}_{\#} )}{60} \qquad [ 9 ]$$

Where P_{#}is defined as the number of pulses per electrode pair, D represents the delay between cycles, εₚ is the total number of electrode pair combinations employed in the therapy, C_{#}is the number of cycles, and f is the pulse delivery rate (90 pulses per minute).

To determine and closeness of fit and validate the numerical model, the numerically calculated initial and final electrical current for each pulsing scheme was compared to the experimentally measured initial and final current. Further, the temperature at each electrode was calculated and compared to the experimentally measured temperatures at each electrode.

### Numerical Model Validation

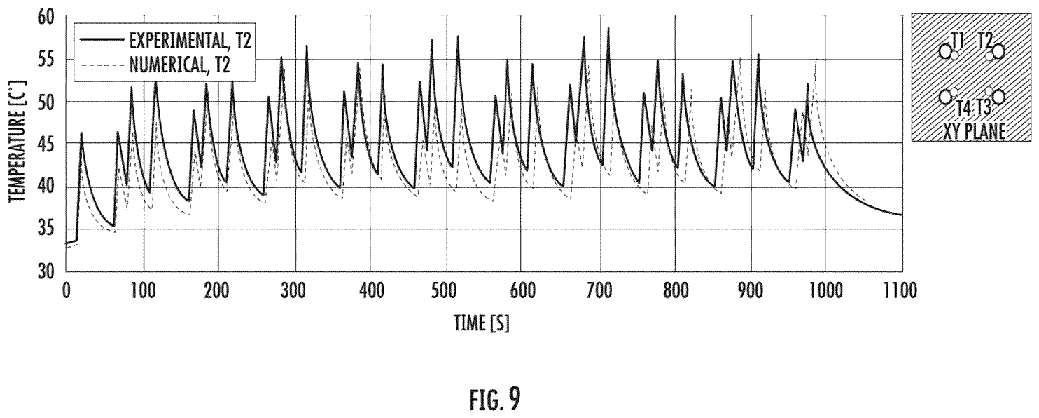

The numerical model current was determined by integrating the normal current density over the sink electrode. Percentage error calculations were performed to compare the numerically calculated currents and average experimentally measured currents at the start and end of treatments. Further, temperature measured at each electrode was compared to the numerical model via root mean square error (RMSE) calculation, also shown in Table 4. FIG. 9 illustrates the closeness in fit from a thermal perspective.

TABLE 4
Comparison between the numerical model and actual experimental
results for the initial and final current of each pulsing scheme:

| | Percent | Percent | |
|---|---|---|---|
| | Error | Error | RMSE |
| | Initial | Final | of |
| | Current | Current | Temperature |
| Pulsing Scheme | [%] | [%] | [° C.] |
| Conventional Paradigm-Exp. | 5.04 | 0.19 | 8.61 |
| Conventional Paradigm-Model | | | |
| 1 pulse cycles, 0 s delay-Exp. | 1.24 | 2.47 | 6.22 |
| 1 pulse cycles, 0 s delay-Model | | | |
| 5 pulse cycles, 0 s delay-Exp. | 1.10 | 10.31 | 8.83 |
| 5 pulse cycles, 0 s delay-Model | | | |
| 10 pulse cycles, 0 s delay-Exp. | 7.23 | 9.74 | 9.16 |
| 10 pulse cycles, 0 s delay-Model | | | |
| 10 pulse cycles, 5 s delay-Exp. | 5.74 | 4.60 | 9.66 |
| 10 pulse cycles, 5 s delay-Model | | | |
| 10 pulse cycles, 10 s delay-Exp. | 6.93 | 4.37 | 6.02 |
| 10 pulse cycles, 10 s delay-Model | | | |

### Statistical Analysis

A total of six livers were used, with 6-8 treatments performed per liver (total N=42). Data are presented as mean values ± standard deviation of the mean. A one-way analysis of variance (ANOVA) was used to determine whether different pulsing paradigms yielded an effect for the parameter of interest. For parameters in which the ANOVA showed an effect, a secondary Tukey's test was performed to determine which pulsing paradigm(s) were significantly different from others. All statistical analysis was performed within JMP® Pro version 14.0.0 (SAS Institute Inc., Cary, NC).

### Results

### Perfused Whole Organ Tissue Testing

FIGS. 10A-F display representative images of the cross-sectional tissue samples for each pulsing paradigm tested, along with the corresponding ratio percentage of thermal damage to IRE ablation. Each pulsing paradigm in this example involved conventional EPAP. FIG. 10G shows the percentage ratio of white tissue coagulation area to IRE treatment zone area for each pulsing scheme. These measurements illustrated that 20.59±6.48% of the ablation presented white tissue coagulation for the “1 pulse cycle, 0 s delay” pulse scheme, whereas all other groups demonstrated less than 5% white tissue coagulation associated with the ablation (p<0.0001, all pairwise comparisons). Similarly, FIGS. 10H and 10I express average IRE treatment zone area and white tissue coagulation area, respectively. As shown in FIG. 10H, the IRE treatment zone across the different protocols remained relatively about the same: Conventional (see also FIG. 10A), 1 pulse cycle with 0 s delay (see also FIG. 10B), 5 pulse cycle with 0 s delay (see also FIG. 10C), 10 pulse cycle with 0 s delay (see also FIG. 10D), 10 pulse cycle with 5 s delay (see also FIG. 10E), and 10 pulse cycle with 10 s delay (see also FIG. 10F). No statistical significance was found between IRE treatment zone area for any pulse schemes evaluated. As shown in FIG. 10I, the amount of thermal damage as measured by white tissue coagulation was different across the different protocols in that less white tissue coagulation was seen as the number of cycles increased and/or as the delay increased, with the 10 pulse cycle with 10 s delay showing the least amount of tissue damage. However, the “1 pulse cycle, 0 s delay” pulse scheme produced 2.53±0.48 cm² of white tissue coagulation while all other groups demonstrated less than 0.5 cm² white tissue coagulation associated with the IRE treatment zone (p<0.0001, all comparisons).

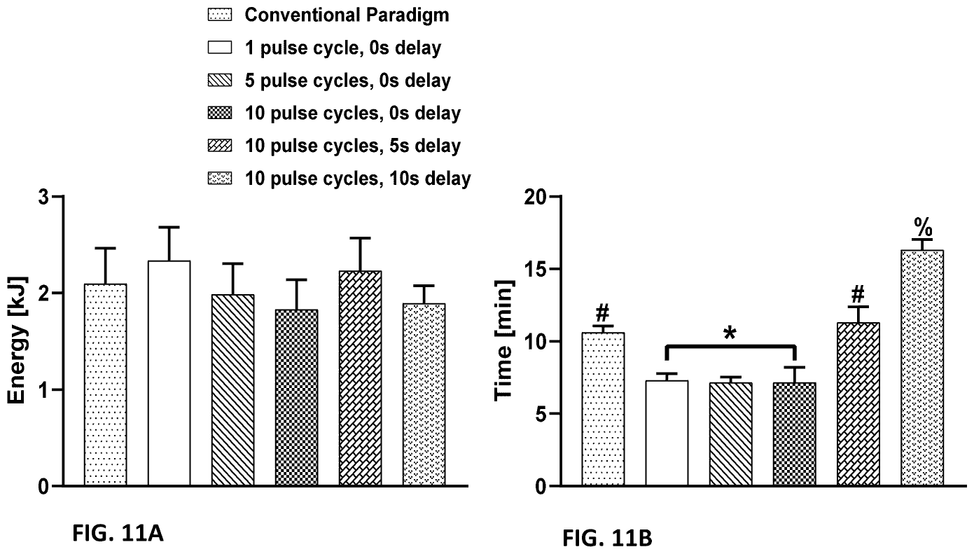

FIG. 11A illustrates the energy delivered for each pulse paradigm tested. No statistical significance was found between experimental groups in energy deposition, thus the energy deposited was about the same across the groups. The “1 pulse cycle, 0 s delay” group induced an average energy of 2.33±0.35 kJ, while the conventional pulse scheme yielded a 2.09±0.37 kJ on average. The “5 pulse cycle, 0 s delay”, “10 pulse cycle, 0 s delay”, and the “10 pulse cycle, 10 s delay” averages of 1.98±0.32 kJ, 1.88±0.34 kJ and 1.89±0.23 kJ, of energy respectively. The “10 pulse cycle, 5 s delay” pulse scheme generated an average energy of 2.14±0.38 kJ.

FIG. 11B expresses the measured treatment duration. Here, all pulse cycle groups with a zero second delay were statistically similar in treatment time and further displayed shorter treatment times on average (approximately 7 minutes for all 0 s delay pulse schemes; p<0.0001 all pairwise comparisons, group *) than other pulse schemes. The conventional paradigm and “10 pulse cycle, 5 s delay” group were found to be statistically similar compared to other groups (10.61±0.46 min, and 11.29±1.11 min; p<0.0001 all pairwise comparisons, group #). Lastly, the treatment time for the “10 pulse cycle, 10 s delay” group was the longest on average and statistically greater than all other groups (16.29±0.76 min, p<0.0001 all pairwise comparisons, group %).

### Numerical Model Results

Here we assumed that the minimum electric field required to induce cell death with HFIRE (the lethal threshold) could be determined by comparing the measured ablation area with those predicted from the numerical model (see C. B. Arena, C. S. Szot, P. A. Garcia, M. N. Rylander, and R. V. Davalos, “A three-dimensional in vitro tumor platform for modeling therapeutic irreversible electroporation,” Biophys. J., vol. 103, no. 9, pp. 2033-2042, 2012; J. W. Ivey, E. L. Latouche, M. B. Sano, J. H. Rossmeisl, R. V. Davalos, and S. S. Verbridge, “Targeted cellular ablation based on the morphology of malignant cells,” Sci. Rep., vol. 5, pp. 1-17, 2015; and E. M. Wasson, J. W. Ivey, S. S. Verbridge, and R. V. Davalos, “The feasibility of enhancing susceptibility of glioblastoma cells to IRE using a calcium adjuvant,” vol. 45, no. 11, pp. 2535-2547, 2018). The electric field that yielded the closest matching volumetric dimensions would be designated as the lethal threshold. FIG. 10H illustrates the area of tissue within the electric field threshold of 601 V/cm for each pulse paradigm. This threshold was most similar to the experimentally measured IRE areas for each pulse paradigm (˜12 cm²). There was no more than 3% difference, between any of the pulsing schemes 400, 600, 800, or 1000 V/cm. A volume integration for tissue greater than 601 V/cm was also performed, which revealed approximately 25 cm³ of tissue with an electric field threshold of 601 V/cm.

FIG. 10I illustrates the thermally damaged tissue area for each pulse scheme. The “1 pulse cycle, 0 s delay” pulse group portrayed 1.68 cm² of tissue experiencing white tissue coagulation (Ω=2.3), well within one standard deviation of the experimental data (difference of 0.72 cm²). The conventional pulsing protocol yielded at 0.65 cm² of tissue experiencing white tissue coagulation. The “10 pulse cycle, 10 s delay” pulse cycle sequence illustrated the least, with an area of 0.005 cm², or a 99.3% reduction of thermal damage area in comparison to the conventional pulse scheme. As a result, the practitioner is able to locate electrode(s) closer to critical non-target structures and thus in a more desired location for treatment and/or expand the treatment area because of the reduction of any thermal effect as compared to the conventional pulse scheme. Each multi-cycle pulsing paradigms illustrated less area thermal damage on average than the single-cycle scheme (“1 pulse cycle, 0 s delay” and the conventional pulse scheme).

The percentage ratio of thermal injury area to IRE ablation area was also evaluated numerically. These numerical results were within one standard deviation of the experimentally measured results and indicated that as much as 13.14% of the “1^{st} pulse cycle, 0 s delay” ablation area would present white tissue coagulation. FIG. 10G illustrates these findings in detail.

Further, a direct comparison between the conventional and enhanced EPAPs was performed to identify any significant differences in IRE or white tissue coagulation areas. The results indicate essentially no changes to the IRE area (FIG. 12A) and a slight reduction in thermal damage area (FIG. 12B) for all pulse paradigms. Overall, the incorporation of cycled pulsing, with an enhanced EPAP could reduce the surface area and volumetric thermal damage as much as 13.6% in comparison to the conventional pulse paradigm, while maintaining the effective electric field thresholds within 2.1%. The percentage ratio of thermal damage area to IRE treatment zone area is shown in FIG. 12C, which shows a similar overall reduction in thermal damage by using cycled pulsing with enhanced EPAP.

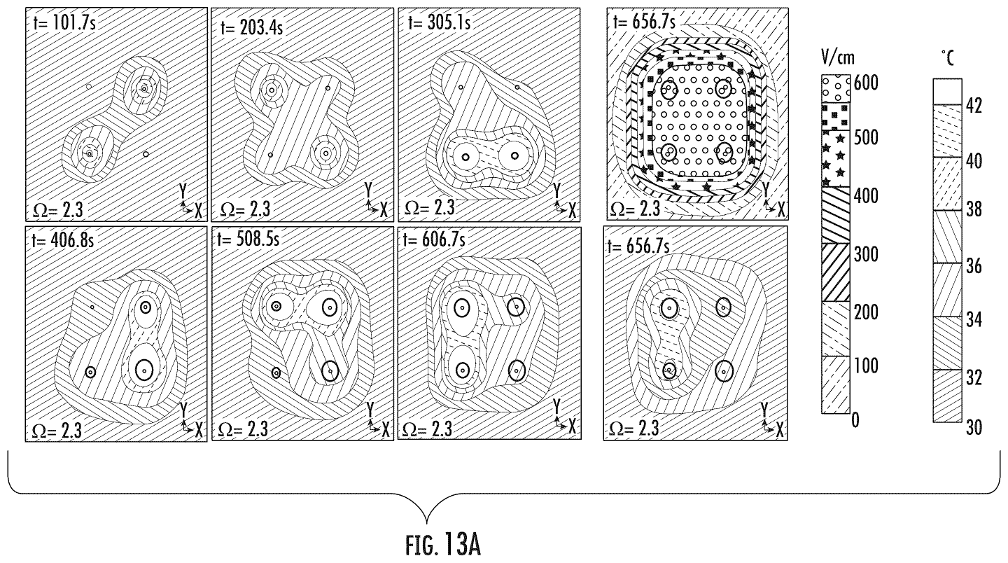

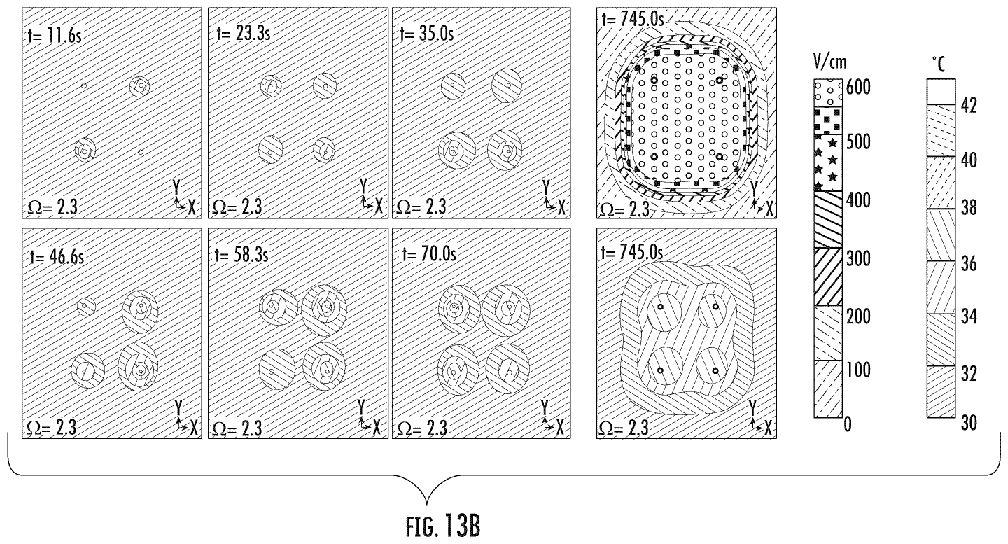

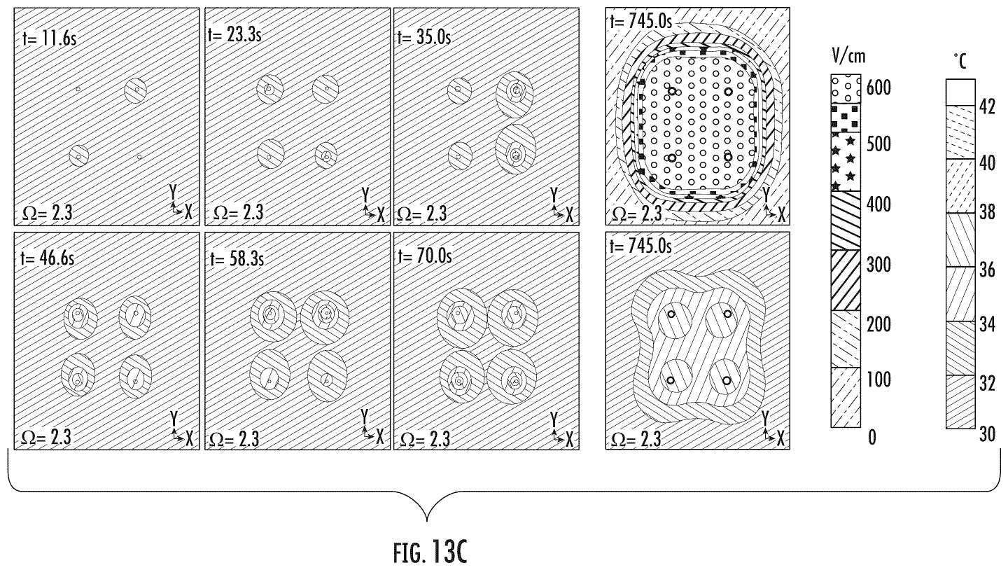

FIGS. 13A-C shows the numerical results for the comparison of (a) the conventional sequence using a conventional EPAP sequence too (FIG. 13A), (b) a 10 pulse cycle, 5 s delay cycled pulse paradigm (FIG. 13B) using a conventional EPAP sequence (i.e., the same conventional EPAP sequence as in FIG. 13A), and (c) the same cycled pulse paradigm model as in FIG. 13B using an enhanced EPAP sequence (FIG. 13C) (enhanced EPAP sequence meaning here that the same pulse protocol of FIG. 13B is applied, but that no single electrode is activated immediately consecutively more than twice). As shown, the amount of thermal damage with the conventional protocol (FIG. 13A) was larger (see the larger areas circled around the electrodes) than that of the other two protocols (FIGS. 13B and 13C). Analysis was performed across the plane, at the midpoint of the electrodes within the 3D model. A series of time-lapse images illustrates the thermal distribution at the first cycle of each electrode pair. The farthest right panels illustrate the thermal distribution and effective electric field at the completion of each treatment. Further, the boundary at which white tissue coagulation (Ω=2.3) occurs is overlaid and outlined in bold. The thermal distribution throughout and completion of treatment illustrates an asymmetric thermal distribution for the conventional pulse paradigm, while both cycled pulsing schemes illustrated uniform temperature distributions and smaller thermal damage volumes. The protocol of FIG. 13A for the conventional treatment can be correlated with the “conventional” treatment results (first set of results) shown in FIGS. 10G-I, 11A-B and 12A-C. The protocols of FIGS. 13B-C for the 10 pulse cycle with 5 s delay can be correlated with the “10 pc/5 s delay” results (second set of results from left) of FIGS. 10G-I, 11A-B and 12A-C.

### Discussion

The inventors studied the effects of varying pulsing paradigms on induced current, resultant temperature, and treatment zone size were investigated for multi-electrode (4-electrode configuration) IRE treatments. The desirable effects of IRE treatments are not dependent on thermal events, but thermal damage can still occur due to Joule heating. The restructuring of the pulse delivery mechanism with the intent to redistribute the delivered energy uniformly across all electrodes and reduce the successive on (energized) time that any active electrode may experience throughout treatment could improve IRE treatment by limiting thermal tissue damage, preventing generator crashes, and enhancing energy delivery to the tissue.

Finite Element Analysis provided insight into the effects of cycled pulsing on the temperature distribution, and thermal injury distribution. More specifically, it was found that cycled pulsing patterns mitigate the overall temperature rise and thermal damage in comparison to single cycle pulsing schemes. Further, the work reported here illuminated the potential importance of electrode-pair activation-order within the pulsing scheme, as any reduction in potential thermal damage is critical for clinicians operating in sensitive regions.

The experimental results utilizing a perfused organ model as a test bed demonstrated that the use of cycled pulse paradigms can reduce the effects of Joule heating while effectively maintaining and, in some cases, improving the IRE treatment zone dimensions in comparison to treatments administered with a conventional pulse scheme. We have verified the effects of using pulse cycle patterns on electrical current, tissue temperature, treatment zone size, and total treatment time for several pulsing schemes. The results indicated that the “5 pulse cycle, 0 s delay” pulsing scheme provided the largest ablation areas while maintaining a relatively low electrical current output and thermal damage index on average. By comparison, the conventional pulsing scheme yielded the second smallest ablation area, a longer treatment time, and a slightly larger electrical current output on average. Regardless, all of the multi-cycle pulse paradigms evaluated yielded a lower current output and larger ablation area than both single-cycle pulse schemes. Further, experimental testing established that inter-cycle delays become a critical factor in determining statistical significance between pulse schemes.

It should be noted that the numerical models described throughout this work define tissue temperature at 30° C. to match the perfused organ model settings, rather than a more representative tissue temperature during surgery (˜35° C.). Models were developed to evaluate the thermal damage volumes at these elevated temperatures. These models revealed larger thermal injury volumes (<2.2 cm³ difference), however, the differences in thermally damaged area for cycled pulse schemes in comparison to the conventional pulse paradigm remained similar.

Here, the inventors tested the effects of using several pulse-timing paradigms on electrical current, tissue temperature, and tissue treatment size. This work successfully shows that cycled pulsing schemes reduce the electrical current, increase the treatment zone size, and ultimately maintain a low tissue temperature in comparison to conventional pulsing schemes in an ex vivo perfused porcine liver model. As demonstrated, cycled pulsing patterns can be an effective tool for enhancing the efficacy of IRE application in clinical practice and would be expected to lead to better overall outcomes for patients.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of treating a tissue, the method comprising:
disposing a plurality of electrodes in a target region of the tissue to be treated; and
activating a generator to deliver electrical pulses to the target region via the plurality of electrodes;
wherein the activating includes delivering trains of electrical pulses between pairs of electrodes in a cyclic manner, wherein a single cycle includes a train of electrical pulses being delivered between each unique pair of electrodes of the plurality of electrodes in a consecutive order and cycles are repeated until a total number of desired pulses per unique electrode pair is achieved; and
wherein the activating is performed such that no single electrode is activated more than two consecutive times.

2. The method of claim 1, wherein the activating is further performed such that:
the total number of desired pulses to be delivered between each unique pair of electrodes is equal to 70-90 pulses;
a number of electrical pulses within each train of electrical pulses delivered between each unique pair of electrodes is equal to 10 pulses; and
there is a delay between each cycle of 10 seconds.

3. The method of claim 1, wherein the tissue includes a prostate tissue or a pancreatic tissue.

4. The method of claim 1, wherein the activating is performed such that there is a 0-10 second delay between pulses of a single train.

5. The method of claim 1, wherein the activating is performed such that there is a 0-10 second delay between trains of a single cycle.

6. The method of claim 1, wherein each of the electrical pulses comprises a burst of pulses.

7. The method of claim 1, wherein electrical energy is strategically distributed to sub-regions within the outline of the target region to treat tissue while mitigating one or more of thermal effects, thermal damage, potential for Joule heating, or delivery of electric current to tissue of the target region.

8. The method of claim 7, wherein the thermal effects or thermal damage are evidenced by an amount of white tissue coagulation.

9. The method of claim 1, wherein a ratio of thermally damaged tissue area to ablation area is less than 5%.

10. The method of claim 1, wherein same and/or different sub-regions of the target region are treated consecutively.

11. The method of claim 10, wherein the delivering of the electrical pulses causes electroporation based therapy, electroporation, irreversible electroporation, reversible electroporation, electrochemotherapy, electrogenetherapy, supraporation, and/or high frequency irreversible electroporation, or combinations thereof.

12. A method of treating a tissue, the method comprising:
delivering a first subset of electrical pulses via a first pair of electrodes disposed within the tissue, wherein the first subset of electrical pulses is less than a total number of desired pulses to be applied by the first pair of electrodes;
delivering a second subset of electrical pulses via a second pair of electrodes disposed within the tissue, wherein the second subset of electrical pulses is less than a total number of desired pulses to be applied by the second pair of electrodes, wherein the second pair of electrodes includes at least one different electrode from the first pair of electrodes; and
delivering additional subsets of electrical pulses via the first and second pairs of electrodes sequentially until the total number of desired pulses for each pair is delivered thereby resulting in a reduction of a concentration of electrical energy applied to the tissue per unit to time or a reduction of an electrical field applied to the tissue per unit of time;
wherein the total number of desired pulses to be delivered between each of the first pair and second pair of electrodes is equal to 70-90 pulses; and
wherein the first subset of electrical pulses and the second subset of electrical pulses each comprise 10 pulses.

13. The method of claim 12, wherein the tissue includes a prostate tissue, a pancreatic tissue, a uterine fibroid, a renal tissue, a colon tissue, a renal tissue, a liver tissue, or a bile duct tissue.

14. The method of claim 12, wherein there is a 0-10 second delay between pulses of a single subset of electrical pulses.

15. The method of claim 12, wherein there is a 0-10 second delay between subsets of electrical pulses.

16. The method of claim 12, wherein each electrical pulse is a burst of pulses.

17. The method of claim 12, wherein electrical energy is strategically distributed to sub-regions within the outline of the target region to treat tissue while mitigating one or more of thermal effects, thermal damage, potential for Joule heating, or delivery of electric current to tissue of the target region.

18. The method of claim 17, wherein the thermal effects or thermal damage are evidenced by an amount of white tissue coagulation.

19. The method of claim 12, wherein the delivering of the electrical pulses causes electroporation based therapy, electroporation, irreversible electroporation, reversible electroporation, electrochemotherapy, electrogenetherapy, supraporation, high frequency irreversible electroporation, or combinations thereof.

20. A method of irreversibly electroporating a target tissue, the method comprising:
electrically coupling a plurality of electrodes to a generator;
disposing the plurality of electrodes near the target tissue; and
activating the generator to deliver electrical pulses to the target tissue via the plurality of electrodes, wherein:
the electrical pulses are delivered in a cycled pulse sequence such that a total number of electrical pulses delivered between the plurality of electrodes is divided into subsets of electrical pulses;
the plurality of electrodes are activated in a sequential order to deliver the subsets of electrical pulses to define a pulse cycle; and
additional pulse cycles are repeated until the total number of desired pulses are delivered, wherein an order that the plurality of electrodes are activated in a given pulse cycle is altered in a next pulse cycle.
